(19) United States
(12) Patent Application Publication
(10) Pub. No.: US 2012/0150006 A1
(43) Pub. Date: Jun. 14, 2012

(54) STIMULI RESPONSIVE MEMBRANE

(75) Inventors: Laurent Lavanant, Evian (FR); Harm-Anton Klok, Ecublens (CH)

(73) Assignee: SENSILE PAT AG, Haegendorf (CH)

(21) Appl. No.: 13/389,823

(22) PCT Filed: Aug. 10, 2010
(86) PCT No.: PCT/IB10/53608

§ 371 Date: Feb. 10, 2012

(30) Foreign Application Priority Data
Aug. 10, 2009 (EP) 09167572.8

(51) Int. Cl. A61B 5/1468; B05D 5/00; G01N 33/48; B82Y 35/00

(52) U.S. Cl. 600/347; 422/68.1; 427/244; 977/755; 977/904

(57) ABSTRACT

There is provided a glucose responsive membrane comprising a nanoporous support substrate and a coating of a glucose responsive hydrogel attached to a surface of the nanoporous substrate. There are also provided methods for the preparation of the glucose responsive membrane and a medical device for the monitoring or regulation of glucose levels in a patient comprising the membrane.

The present invention relates to the field of porous membranes, more particularly to the design and manufacture of porous membranes which are responsive to external stimuli, and to blood analyte monitoring and drug delivery devices comprising the membranes, in particular for the monitoring of glucose and for the treatment of patients with diabetes.

The regular or continuous measurement of an analyte concentration is necessary in the control or therapy of many conditions, such as diabetes. For instance, diabetic patients may require measurement of their blood glucose level several times a day, in order to appropriately adapt the administration of insulin. More measurements of the blood glucose level allow for drug administration regimes which regulate the blood glucose level of the diabetic patient more precisely, i.e. the fluctuations of the blood glucose level may be kept within a physiological range. Hence, it is crucial for a successful treatment of diabetic patients to obtain accurate, undelayed, and continuous information about the blood glucose level.

Various different medical devices have been proposed for the monitoring of blood glucose levels. Most conventionally used blood glucose monitors make use of chemical test strips which work on electro-chemical principles, whereby the patient withdraws a drop of blood for each measurement, generally requiring uncomfortable finger pricking methods. In order to avoid the pain caused by finger pricking and to allow more frequent, or continuous, control of glycaemia a variety of implantable sensors, including transdermal or subcutaneous sensors, are being developed for continuously detecting and/or quantifying blood glucose values. Glucose sensors for frequent or continuous glucose monitoring based on electrochemical, viscosimetric, or optical sensors have been widely investigated.

Different medical devices intended for the treatment of patients with diabetes have previously been described: Separate glucose sensors (e.g. electrochemical, viscosimetric, or optical sensors); separate medication delivery devices (e.g. insulin pumps and insulin pens); as well as so-called closed loop systems integrating glucose sensor and medication delivery, which ideally mimic the function of the pancreas, i.e. medication capable of controlling blood glucose level is released subject to blood glucose concentration.

Although a number of closed loop systems have been investigated, as yet none has been successfully developed for practical usage in patients.

To date most development in closed loop systems has related to so-called two port systems which consist of at least two separated units, connected through an electronic interface. For instance in patent application US 2006/0224141 there is described a system, in which the analyte monitoring unit is separated from the medication infusion unit. The analyte sensor is based on using electrodes in order to determine a change in electric resistance subject to a change of the analyte concentration. A semi-closed loop system is described in patent application WO 03047426A1, where an at least partially implanted glucose sensor is in communication with an injection pen, whereas the user can adjust the dose to be injected based on the glucose concentration measured by the glucose sensor. Such two port systems require the insertion of catheter or needle for insulin delivery at a different skin site to that of the glucose sensor. This spatial separation results in patient discomfort and reduces patient acceptability of the closed loop system.

WO 89/01794 discloses an implantable glucose sensor for a one port integrated drug delivery system. The sensor includes a liquid infusate, which is put under pressure and flows through a catheter. One section of the catheter contains a microporous membrane, where the concentration of the glucose present in the infusate is equilibrated with a response time between several minutes up to one hour. The equilibrated infusate then flows through a chemical valve which consists of a hydrogel matrix containing concanavalin A, and dextran molecules. The matrix in the chemical valve changes its solute permeability subject to the glucose concentration present in the infusate, thus regulating the amount of infusate flowing into the body of a patient.

When the system, as disclosed in WO 89/01794, is employed to solely monitor the concentration of glucose in the surrounding medium, the catheter contains an additional glucose sensor, such as an enzyme electrode, a fuel cell, or an affinity sensor, whereas the chemical valve is not present. Further proposed is a stand-alone sensor, in which the pressure in the infusate is determined before and after the infusate has passed the chemical valve, whereas the pressure-drop across the chemical valve is inversely proportional to the glucose concentration in the equilibrated infusate.

In order to control the blood glucose level in a patient with diabetes, it is necessary to obtain results quickly in order to adjust the delivery of drugs. That is why response times of components within the glucose sensor are a crucial factor for a successful drug delivery program. If, as described in WO 89/01794, an equilibration region has a response time of up to one hour, and a hydrogel matrix contained in a chemical valve has an additional response time, the drug administration is adjusted to a blood glucose value that is no longer present in the patient, and thus the regulation of the patient's blood glucose level will not be optimal.

Further where, as in WO89/0174, the hydrogel matrix is in a fluent state, i.e. new components (such as dextran molecules) that arrive with fresh infusate replace components that are washed away with the infusate into the patient's body, then components that do not contribute to the treatment, or even are toxic such as is the case for concanavalin A, may enter the patient's body. Moreover, the matrix is likely to change characteristics over time, as the replacement of new components may not take place in an evenly distributed manner. For instance, clusters are likely to occur at the infusate entry site(s) of the matrix where the infusate with new components arrives at first.

A number of hydrogels have been investigated for potential application in glucose concentration measurement (see for instance: T. Miyata, T. Uragami, K. Nakamae Adv. Drug Deliver. Rev. 2002, 54, 79.; Y. Qiu, K. Park Adv. Drug. Deliver. Rev. 2001, 53, 321.; S. Chaterji, I. K. Kwon, K. park Prog. Polym. Sci. 2007, 32, 1083.; N. A. Peppas J. Drug Del. Sci. Tech. 2004, 14, 247-256). Hydrogels are cross-linked polymeric matrices that absorb large amounts of water and swell. These materials may be physically and chemically cross-linked to maintain their structural integrity. Hydrogels can be sensitive to the conditions of the external environment if they contain active functional groups. The swelling behavior of these gels may be dependent on for instance pH, temperature, ionic strength, solvent composition, or other environmental parameters. These properties have been used to design stimuli responsive or “intelligent” hydrogels such as glucose-sensitive polymeric systems. (see for instance: G. Albin, T. A. Horbett, B. D. Ratner, J. Controlled Release, 1985, 2, 153.; K. Ishihara, M. Kobayashi, I. Shinohara Polymer J. 1984, 16, 625)

Among the different types of hydrogel responsive to glucose which have been proposed, three main types of hydrogel have been investigated:

### Glucose Oxidase-Loaded Hydrogels:

The combination of a pH sensitive hydrogel with the enzyme glucose oxidase (GOD) has been investigated for the design of glucose responsive hydrogels. Glucose is enzymatically converted by GOD to gluconic acid which lowers the pH of the environment. The enzyme GOD has been combined to different types of pH sensitive hydrogels. In general for hydrogels that contain polycations, such as poly(N,N′-diethylaminoethyl methacrylate), the lowering of pH leads to hydrogel swelling due to the protonation of the N,N′-diethylaminoethyl side chain. When a hydrogel swells, molecules diffuse more easily through the hydrogel when compared to the collapsed state. Whereas, if the hydrogel contains polyanions, such as poly(methacrylic acid), the hydrogel swells at high pH value due to electrostatic repulsion among the charges on the polymer chains. After lowering of the pH, the polymer chains of the hydrogel collapse due to the protonation of the methacrylic acid side chains which reduces the electrostatic repulsion between the polymer chains. (Y. Ito, M. Casolaro, K. Kono, I. Yukio J. Controlled Release 1989, 10, 195)

### Lectin-Loaded Hydrogels:

Another approach to design glucose responsive hydrogels consists in combining glucose containing polymers with carbohydrate-binding proteins, lectins, such as Concanavalin A (Con A). The biospecific affinity binding between glucose receptors of Con A and glucose containing polymers leads to the formation of a gel capable of reversible sol-gel transition in response to free glucose concentration. A variety of natural glucose containing polymers have been investigated such as polysucrose, dextran, and glycogen (see for instance: M. J. Taylor, S. Tanna, J. Pharm. Pharmacol. 1994, 46, 1051; M. J. Taylor, S. Tanna, P. M. Taylor, G. Adams, J. Drug Target. 1995, 3, 209; S. Tanna, M. J. Taylor, J. Pharm. Pharmacol. 1997, 49, 76; S. Tanna, M. J. Taylor, Pharm Pharmacol. Commun. 1998, 4, 117; S. Tanna, M. J. Taylor, Proc. Int. Symp. Contr. Rel. Bioact. Mater. 1998, 25, 737B.; S. Tanna, M. J. Taylor, G. Adams, J. Pharm. Pharmacol. 1999, 51, 1093). Additionally, some synthetic polymers with well defined saccharide residues such as poly(2-glucosyloethyl methacrylate) (PGEMA) have been investigated. (K. Nakamae, T. Miyata, A. Jikihara, A. S. Hoffman J. Biomater. Sci. polym. Ed. 1994, 6, 79.)

### Phenylboronic Acid Based Hydrogels:

The fabrication and handling of glucose responsive hydrogels that incorporate proteins is difficult due to the instability of biological components. As an alternative approach, investigation has been made on synthetic hydrogels that contain phenylboronic acid (PBA) moieties.

Phenylboronic acid and its derivatives form complexes with polyol compounds, such as glucose, in aqueous solution. It has been shown that these Lewis acids can reversibly bind the cis-1,2- or -1,3-diols of saccharides covalently to form five- or six-membered rings (C. J. Ward, P. Patel, T. D. James, Org. Lett. 2002, 4, 477.) The complex formed between phenylboronic acid and a polyol compound can be dissociated in the presence of a competing polyol compound which is able to form a stronger complex with the phenylboronic acid. Following this idea, the competitive binding of phenylboronic acid with glucose and poly(vinyl alcohol) has been investigated for the construction of a glucose-sensitive material. For example, the competitive binding of the PBA moieties of poly(N-vinyl-2-pyrrolidone)-co-poly(3-(acrylamido)phenylboronicacid) copolymer with glucose and poly(vinyl alcohol) (S. Kitano, Y. Koyama, K. Kataoka, T. Okano, Y. Sakurai, J. Controlled Release 1992, 19, 161).

In aqueous medium Phenylboronic acid compounds exist in equilibrium between an uncharged and a charged form. Only the charged phenylborates form a stable complex with glucose, whereas unstable complex are obtained between glucose and the uncharged form. When the concentration of glucose increases, the total amount of charged PBA moieties increases and the number of uncharged groups decreases which has a dramatic effect on the solubility of the polymer in water (K. Kataoka, H. Miyazaki, T. Okano, Y. Sakurai, Macromolecules 1994, 27, 1061). This change in solubility has been investigated for the development of a glucose sensitive system. For example, PBA has been copolymerized with temperature sensitive polymer such as N-isopropylacrylamide (NIPAM) in order to obtain a polymer with a glucose sensitive low critical solution temperature (LOST) (T. Aoki, Y. Nagao, K. Sanui, N. Ogata, A. Kikuchi, Y. Sakurai, K. Kataoka, T. Okano, Polym. J. 1996, 28, 371). In this system a change in glucose concentration induces a change in the LOST of the hydrogel which, at a fixed temperature, will induce swelling/collapse of the hydrogel matrix.

Despite promising results, the systems described above cannot be used for application in in-vivo monitoring of glucose concentration for two important reasons:

1) Physiological condition: Reversible binding of phenylboronic acid with polyol was not achieved at physiological conditions (temperature, ionic strength and pH values).
2) Selectivity and interfering molecules: The binding of phenylboronic acid is not selective. Indeed, phenylboronic acids can form complexes with any saccharides possessing cis-1,2- or -1,3-diols (such as glucose, fructose and galactose). In healthy individuals glucose is normally present in the range 4-8 mM while fructose and galactose, the most abundant sugars after glucose, are usually present in physiological fluids at sub-mM levels (R. Badugu, J. R. Lakowicz, C. D. Geddes, Analyst 2004, 129, 516). However, phenylboronic acids have a much greater affinity for fructose than glucose, (J. P. Lorand, J. O. Edwards, J. Am. Chem. Soc. 1959, 24, 769) a feature that may affect the accuracy of glucose measurement. Additionally, the presence of lactate is known to interfere with phenylboronic acid based hydrogels.

Some formulations of hydrogels with phenylboronic acid moieties have been recently investigated with the aim to improve the selectivity of the hydrogel and/or the better reversibility at physiological conditions. It has been found that the presence of basic groups, such as amines, in the neighbourhood of the PBA moieties allows the formation of stable complexes with glucose at physiological pH. This is due to basic amino groups which might coordinate to boron atoms to stabilize the tetrahedral form of the boronic acid moiety (see for instance: Kikuchi A, Suzuki K, Okabayashi O, Hoshino H, Kataoka K, Sakurai Y, Okano T: Glucose-sensing electrode coated with polymer complex gel containing phenylboronic acid. Analytical Chemistry 1996, 68, 823-828).

In another formulation, presented by Pritchard in 2006 (G. J. Worsley, G. A. Tourniaire, K. E. S. Medlock, F. K. Sartain, H. E. Harmer, M. Thatcher, A. M. Horgan, J. Pritchard Clinical Chem. 2007, 53, 1820-1826, a tertiary amine monomer (N-[3-(dimethylamino)propyl]-acrylamide) was copolymerized with 3-acrylamidophenylboronic acid to give a glucose responsive hydrogel with a specific affinity for glucose. In this case, an increase of the glucose concentration induces a contraction of the gel. The most probable explanation for the observed contraction is cross-linking of two neighboring boronic acid receptors with favorable stereochemistry by glucose to give a bis-boronateglucose complex. A film of this glucose responsive hydrogel has been loaded with light sensitive crystals of AgBr to design a holographic glucose sensor shown to have ability to detect glucose in human plasma conditions. (See for instance: S. Tanna, T. S. Sahota, J. Clark, M. J. Taylor J. Drug Target. 2002, 10 411; S. Tanna, T. Sahota, J. Clark, M. J. Taylor, J. Pharm. Pharmacol. 2002, 54, 1461).

One solution that has been proposed to provide selectivity to glucose at physiological pH and obviate lactate interference consists of designing PBA derivatives with lower pKa. Following this idea, 2-acrylamidophenylborate (2APB) has been designed and used to fabricate a holographic sensor, which displayed significant response to glucose with little interference from lactate, and with no dependence on pH in the physiological range (see for instance: Yang X P, Lee M C, Sartain F, Pan X H, Lowe C R: Designed boronate ligands for glucose-selective holographic sensors. Chemistry-a European Journal 2006, 12, 8491-8497). This could be explained by the formation of weak B-O intermolecular interactions, conferring a tetrahedral conformation at the boron centre through the neighbouring effect of the ortho group.

WO 2004/081624 describes a class of phenylboronic acid derivatives wherein the phenyl group comprises one or more substituent which via an electronic effect promotes formation of the more reactive tetrahedral geometry about the boron atom. According to WO 2004/081624 the substituent(s) may be electron withdrawing groups which, by mediating their electronic effects through the phenyl ring, promote the formation of the tetrahedral geometry, or may be a substituent capable of forming an intramolecular bond with the boron atom forcing the boronate into the tetrahedral conformation.

PBA derivatives are described having the general structure:

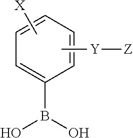

wherein X is an atom or group which, via electronic effect, promotes formation of tetrahedral geometry about the boron atom, Y is a linker, which may be an atom or group which, via electronic effect, promotes formation of tetrahedral geometry about the boron atom and Z is a polymerisable group. Specifically 2-acrylamido-phenylboronic acid and 3-acrylamido-phenylboronic acid were shown to provide significant response to glucose.

The known hydrogel technologies show a number of limitations for use in in-vivo physiological conditions in blood analyte monitoring or regulation applications.

Another important problem encountered with hydrogels that contain proteins such as glucose oxidase or Con A is the leakage of proteins from the gel. Particularly Con A which is known to be a toxic compound. To reduce leakage, it has been proposed to covalently attach proteins to the polymer backbone (see for instance: Tanna S, Sahota T, Clark J, Taylor MJ: A covalently stabilised glucose responsive gel formulation with a Carbopol (R) carrier. Journal of Drug Targeting 2002, 10, 411-418. Tanna S, Sahota T, Clark J, Taylor M J: Covalent coupling of concanavalin A to a Carbopol 934P and 941P carrier in glucose-sensitive gels for delivery of insulin. Journal of Pharmacy and Pharmacology 2002, 54, 1461-1469). However, the covalent attachment of proteins requires some synthetic efforts and may induce modifications of the biological functions of the proteins.

One major constraint to build a sensor for in-vivo applications is that all the components have to be sterilized. Hydrogels that contain proteins cannot be easily sterilized. Indeed, proteins such as glucose oxidase or Con A are sensitive to heat, which means that they cannot be autoclaved, and are denatured by gamma radiations.

An additional constraint for in-vivo sensor applications is that all the components used in the analyte responsive hydrogel should be biocompatible in order to prevent inflammation (acute and chronic) and fibrous encapsulation of the sensor which leads to a loss of sensibility of the sensor. Indeed, the coating itself can also lead to an undesirable response (Beckert W H, Sharkey M M: Mitogenic Activity of Jack Bean (Canavalia-Ensiformis) with Rabbit Peripheral Blood Lymphocytes. International Archives of Allergy and Applied Immunology 1970, 39, 337).

Further, for clinical applications, and especially for closed loop systems in diabetes treatment as described above, ever changing glucose concentrations demand hydrogels that can switch reproducibly and with rapid response onset times on a long-term basis. However, the response of bulk hydrogels upon changes in the environmental glucose concentration occurs too slowly, ranging from tens of minutes to hours.

The volume change process of gels is generally determined by the cooperative diffusion of the polymer in the solvent. As a result, swelling and shrinking of gels is quite slow because the diffusion coefficient of polymers is on the order of 10⁻⁷ cm²/s, while that of water and small ions is on the order of 10⁻⁵ cm²/s. (Katoa N, Gehrke S H: Microporous, fast response cellulose ether hydrogel prepared by freeze-drying, Colloids and Surfaces B: Biointerfaces 2004, 38, 191-196).

The sorption/desorption of solvents by gels is often described by a simple diffusion-controlled process; the polymer network motion of the conventional, non-porous gel is controlled by a diffusional process of polymers and the solvents. Fick's law of diffusion can be applied to the sorption/desorption process of gels and an apparent polymer solvent diffusion coefficient Dp in a polymer-fixed frame of reference is obtained by fitting the following equation to the macroscopic swelling/shrinking data in the case of the flat gel sheet:

$$\frac{{M}_{t}}{{M}_{∞}} = 1 - {\sum }_{n = 0}^{∞} [ \frac{8}{{( 2 n + 1 )}^{2} {π}^{2}} ] exp [ - {( 2 n + 1 )}^{2} {π}^{2} \frac{{D}_{p} t}{{L}^{2}} ]$$

where L is the initial thickness of the flat gel sheet.

The response time of such gels to the environmental change can be reduced by decreasing the characteristic diffusion path length i.e. decreasing L. (Katoa N, Gehrke SH: Microporous, fast response cellulose ether hydrogel prepared by freeze-drying, Colloids and Surfaces B: Biointerfaces 2004, 38, 191-196).

Reducing the hydrogel dimensions may be one potential way of shortening the response time. However reduction of the hydrogel dimensions tends to reduce the hydrogel integrity and may induce modifications in the response of the hydrogel to external stimuli (e.g. reduced swelling, reduced reactivity) which have negative impact on the utility of the hydrogel. For clinical applications, and especially for closed loop systems in diabetes treatment as described above, it is of utmost importance that the integrity, and response characteristics, of the hydrogel be maintained under clinical conditions.

Some systems have been proposed with the aim of maintaining hydrogel integrity. For instance the clamping of the hydrogel polymer in-between dialysis membranes (Kim J J, Park K, Modulated insulin delivery from glucose-sensitive hydrogel dosage forms, Journal of Controlled Release 2001, 77, 39-47), however this results in further increasing the overall response time. It has also been proposed to cast the hydrogel around a mechanical support (M. Tang R Z, A. Bowyer, R. Eisenthal, J. Hubble,: A reversible hydrogel membrane for controlling the delivery of macromolecules, Biotechnology and Bioengineering 2003, 82, 47-53) or hydrogel nanoparticles within a polymer matrix. The polymer casting route does not allow for very thin hydrogel membranes to be fabricated, however, and slow response times will always result. Micro-fluidic concepts have also been investigated whereby hydrogel expansion/contraction depending on glucose concentration provides mechanical opening/closing of a microvalve gate, but with the same limitations (see for instance: Eddington D T, Beebe D J, A valved responsive hydrogel microdispensing device with integrated pressure source. Journal of Microelectromechanical Systems 2004, 13, 586-593).

An object of the invention is to provide an analyte responsive membrane for use in a medical device for the measurement or regulation of analyte levels in a patient which overcomes some or all of the above-described limitations of known analyte responsive hydrogel membranes.

Objects of this invention have been achieved by providing a glucose responsive membrane according to claim 1, and by providing a method for the manufacture of a glucose responsive membrane according to claim 12, and by providing a medical device for the monitoring or regulation of glucose concentration according to claim 17.

Disclosed herein is a glucose responsive membrane, for use in a medical device for the monitoring and/or regulation of blood glucose levels in a patient, comprising a nanoporous support substrate and a thin coating of a glucose responsive hydrogel on the nanoporous support substrate. The glucose responsive hydrogel is strongly attached to a surface of the nanoporous support through covalent bonds or electrostatic interactions. It is preferred that the hydrogel is covalently attached to a surface of the nanoporous substrate.

The glucose responsive hydrogel advantageously comprises phenylboronic acid functional groups, and reversibly changes its three-dimensional configuration and/or surface properties in response to changes in glucose concentration occurring in the medium contacting the hydrogel under physiological conditions.

Dynamic flow properties through the membrane are controlled by the glucose responsive hydrogel coating.

The glucose responsive membrane of the present invention reversibly changes its hydraulic permeability in response to glucose concentration, making it possible to control hydraulic flow rate through the membrane.

The support substrate may be any suitable nanoporous substrate. Preferred substrates include nanoporous polypropylene, polyethylene, cellulose or alumina.

The hydrogel coating preferably comprises a plurality of polymer chains (alternatively referred to herein as polymer brushes), whereby each polymer chain is covalently attached via one chain end thereof to a surface of the nanoporous substrate. At least a portion of the polymer chains are functionalized with phenyl boric acid functional groups.

The hydrogel is preferably formed at the surface of the nanoporous substrate by a controlled surface-initiated polymerisation technique, by which advantageously the thickness of the hydrogel coating and its composition may be closely controlled.

In a preferred embodiment the hydrogel is formed by a controlled surface initiated radical polymerisation process, preferably by surface-initiated atom transfer radical polymerisation (SI-ATRP), from an initiator group covalently attached to the substrate surface.

Advantageously the use of a controlled surface initiated polymerisation process for the formation of the hydrogel coating from the nanoporous substrate surface allows the grafting density, i.e. the distance between grafted polymer chains, as well as the thickness and composition of the hydrogel coating to be accurately controlled. Specifically a thin coating of the hydrogel may be obtained on the nanoporous support substrate with very good hydrogel integrity and stability properties.

Advantageously the hydrogel is attached to a least part of the surface of the internal walls of the pores of the nanoporous substrate, thereby at least partially coating the internal walls of at least a portion of the pores of the nanoporous substrate.

Advantageously the composite membrane of the present invention has good hydrogel integrity and long-term stability.

Advantageously, glucose responsive membranes of the present invention can provide a rapid response time to changes in glucose concentration.

Glucose responsive membranes of the present invention advantageously can provide significant, selective and reversible response to changes in glucose concentration.

The glucose responsive membrane advantageously may further comprise non-fouling functional groups to prevent the non-specific adhesion of proteins, present in interstitial fluid, to the surface of the glucose responsive membrane. In a particular embodiment of the invention a layer of bio-compatible polymer is bound to the glucose responsive hydrogel coating. Advantageously a thin layer of biocompatible polymer may be covalently attached to the glucose responsive hydrogel by a controlled surface initiated polymerisation process.

Also provided herein is a method of preparation of a glucose responsive membrane for use in medical device for the monitoring or regulation of blood glucose levels in a patient, according to claim 17.

There is now provided a medical device for the monitoring and/or regulation of glucose levels in a patient including a glucose responsive membrane, which reversibly changes its hydraulic permeability subject to changes in glucose concentration, said membrane comprising a nanoporous support substrate and a biointerface comprising a glucose responsive hydrogel coating covalently attached to a surface of the nanoporous support substrate. The glucose responsive hydrogel advantageously comprises a polymeric matrix functionalised with phenylboronic acid moieties. Said medical device may optionally include means for administration of a quantity of a drug capable of adjusting glucose concentration, according to a determined glucose concentration.

Advantageously a medical device for the monitoring or regulation of glucose levels is based on mechanical sensing methods. According to a preferred embodiment, the medical device determines glucose concentration in a patient body fluid based on measurement of a flow resistance of a liquid through the glucose responsive membrane.

Further objects, advantageous features and aspects of the invention will be apparent from the claims and the following detailed description and examples, in conjunction with the figures in which:

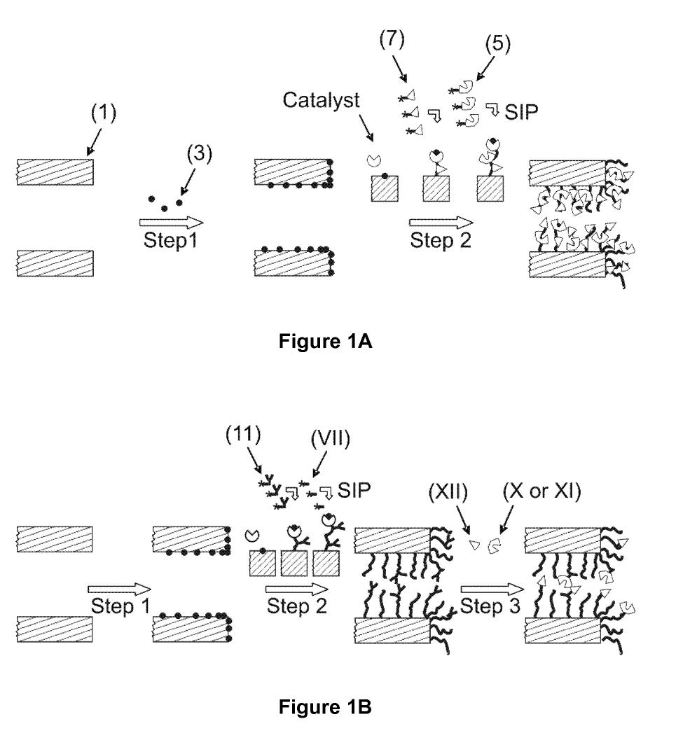

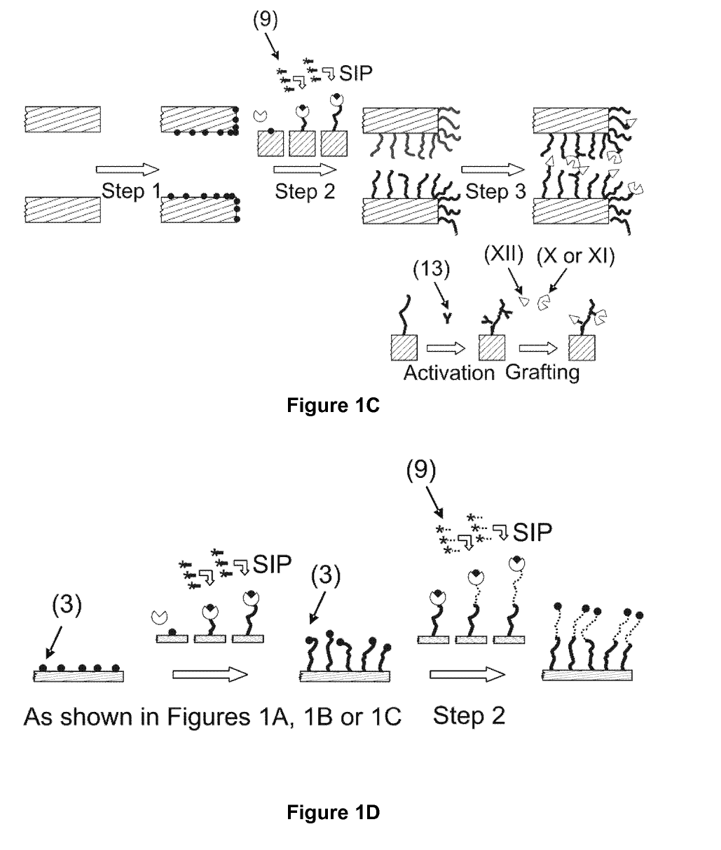

FIG. 1 shows a schematic illustration of selected variants of processes for the preparation of a glucose responsive membrane according to different embodiments of the present invention.

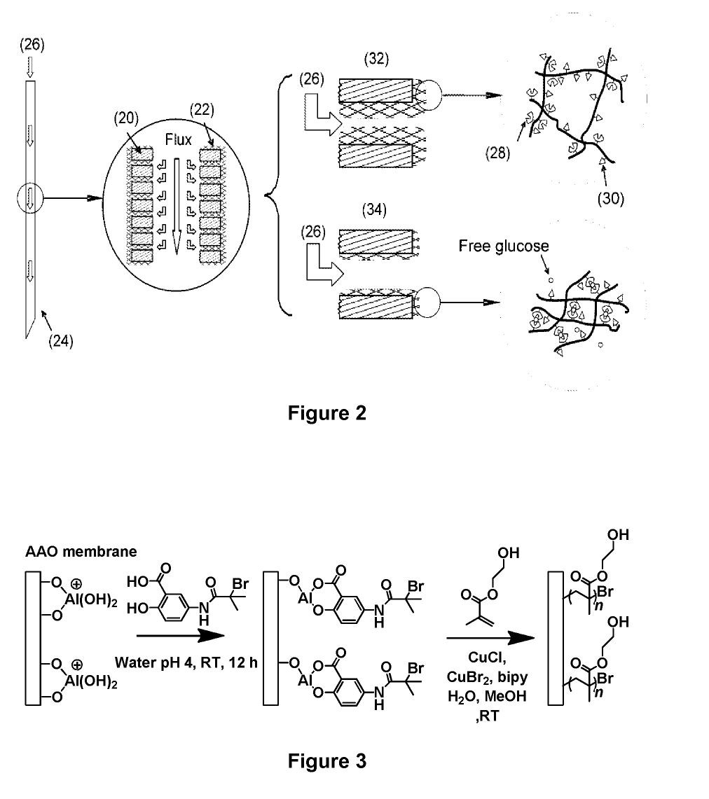

FIG. 2 shows a schematic illustration of part of a device for monitoring or regulating glucose levels comprising a glucose responsive membrane according to an embodiment of the present invention.

FIG. 3 shows a reaction scheme for the formation of a PHEMA polymer brush coating on the surface of a substrate, according to another embodiment of the present invention.

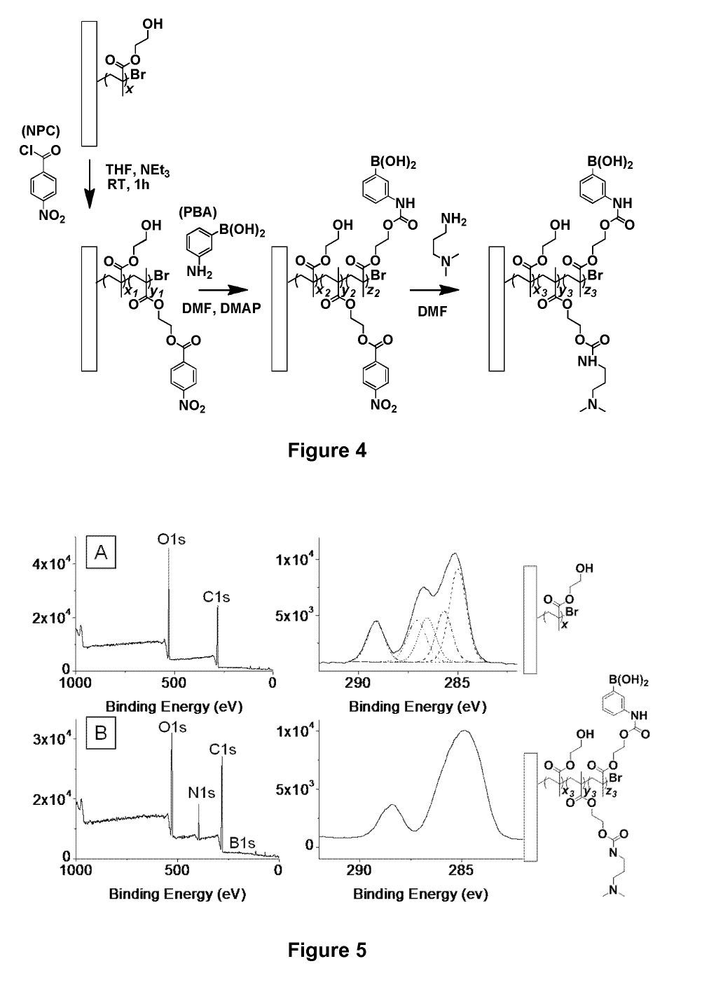

FIG. 4 shows a reaction scheme for the post-modification of a PHEMA polymer brush coating, on the surface of a substrate, with phenyl boronic acid groups, according to another embodiment of the present invention.

FIG. 5 shows XPS survey (left) spectra and XPS C1s (carbon) core level spectra (right) of a membrane according to an embodiment of the invention: (A) AAO membranes coated with PHEMA brushes; (B) AAO membrane coated with PHEMA brushes and functionalized with phenylboronic acid groups.

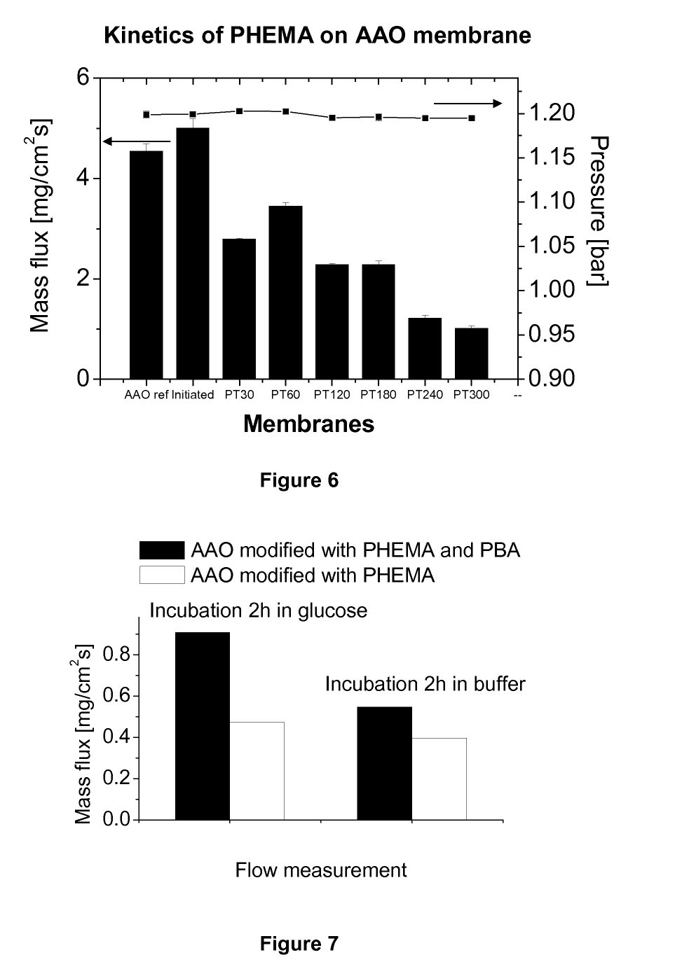

FIG. 6 shows a graphical representation of flow rates for membranes of FIG. 5 modified with PHMA brushes and functionalized with phenylboronic acid groups, prepared with different polymerization times.

FIG. 7 shows a graphical representation of flow rates under 1.20 bar of the membranes of FIGS. 5 and 6, coated with PHEMA brushes (white) and PBA functionalized PHEMA (black), determined after: 2 h incubation in glucose solution at pH 9; followed by 2 h incubation in borate buffer at pH 9.

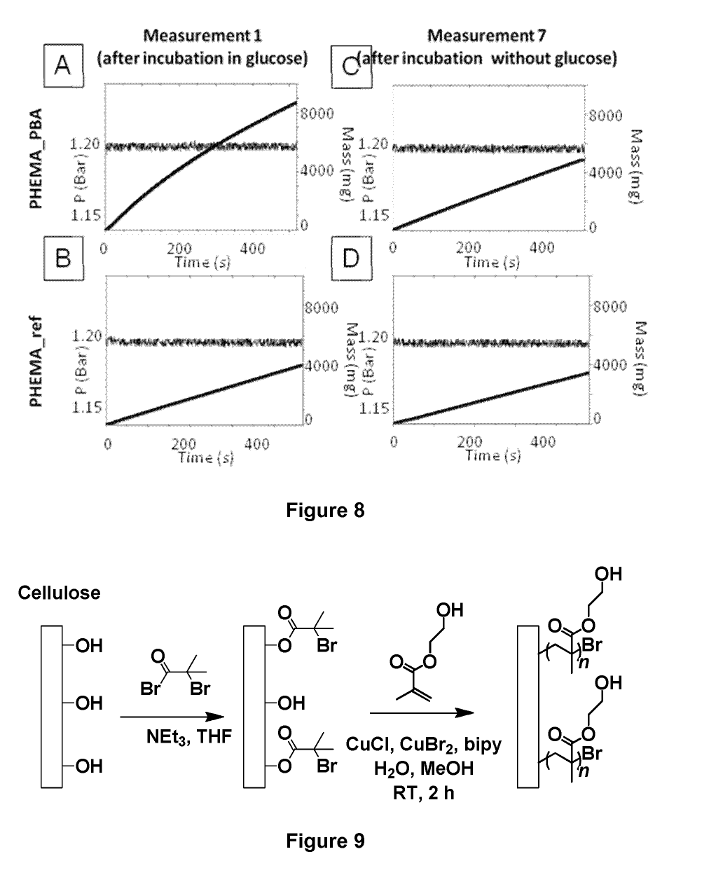

FIG. 8 shows a graphical representation of flow measurement curves of the membranes of FIGS. 5 and 6, coated with coated with PHEMA brushes (A,C) and PBA functionalized PHEMA (B,D), determined after: 2 h incubation in glucose solution at pH 9; and after 2 h incubation borate buffer at pH 9.

FIG. 9 shows a reaction scheme for the formation of a PHEMA polymer brush coating on the surface of a substrate, according to one embodiment of the present invention.

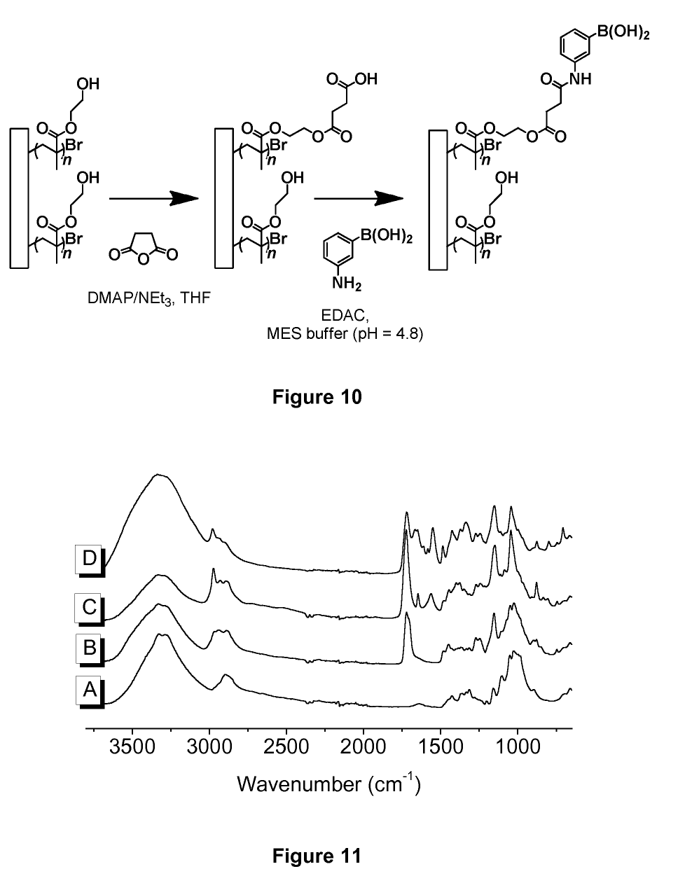

FIG. 10 shows a reaction scheme for the post-modification of a PHEMA polymer brush coating, on the surface of a substrate, with phenyl boronic acid groups, according to one embodiment of the present invention.

FIG. 11 shows ATR-FTIR spectra of a membrane according to one embodiment of the invention: (A) unmodified substrate (SS589/3 substrate); (B) SS589/3 substrate coated with PHEMA brushes; (C)SS589/3 substrate coated with PHEMA brushes and functionalized with carboxylic acid moieties; (D) SS589/3 substrate coated with PHEMA brushes and functionalized with PBA groups.

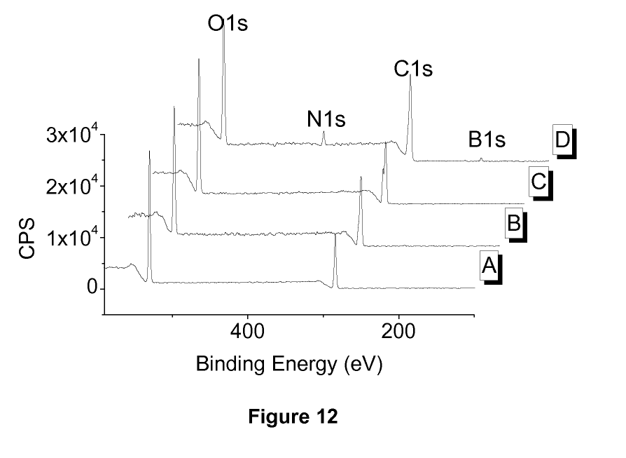

FIG. 12 shows XPS survey spectra of the same membrane: (A) unmodified substrate (SS589/3 substrate); (B) SS589/3 substrate coated with PHEMA brushes; (C) SS589/3 substrate coated with PHEMA brushes and functionalized with carboxylic acid moieties; (D) SS589/3 substrate coated with PHEMA brushes and functionalized with PBA groups.

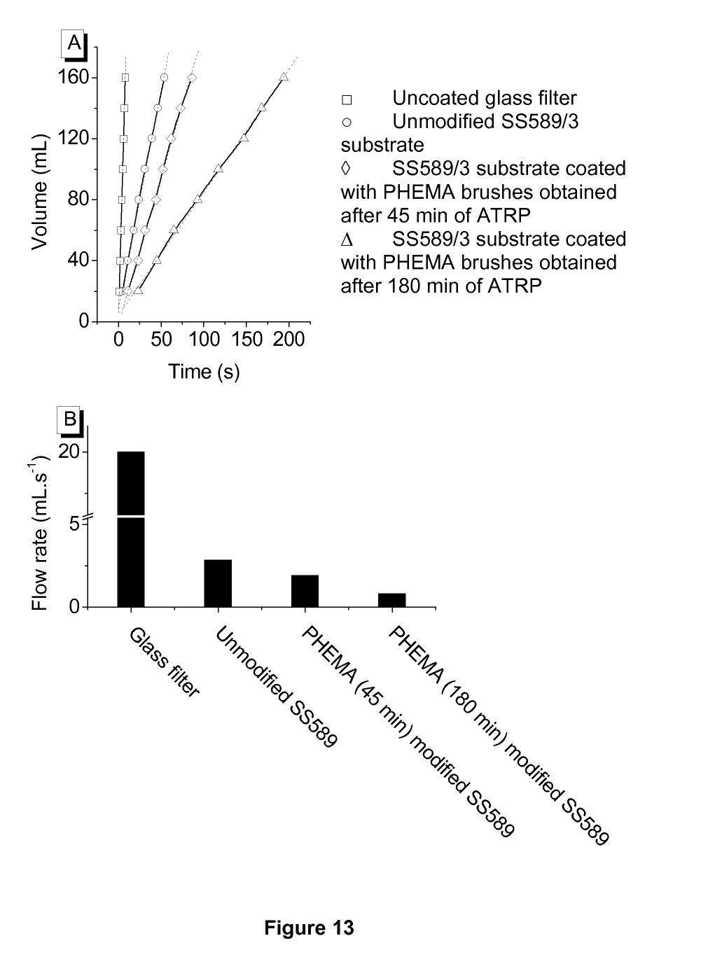

FIG. 13(A) is a graphical representation of fluid flow measurements across the membrane of FIGS. 5 and 6 ((□), unmodified SS589/3 substrate (∘), SS589/3 substrates coated with PHEMA brushes obtained after 45 and 180 min of ATRP (⋄ and Δ espectively))

FIG. 13(B) is a graphical representation of flow rates across the same membrane, as calculated from FIG. 7(A).

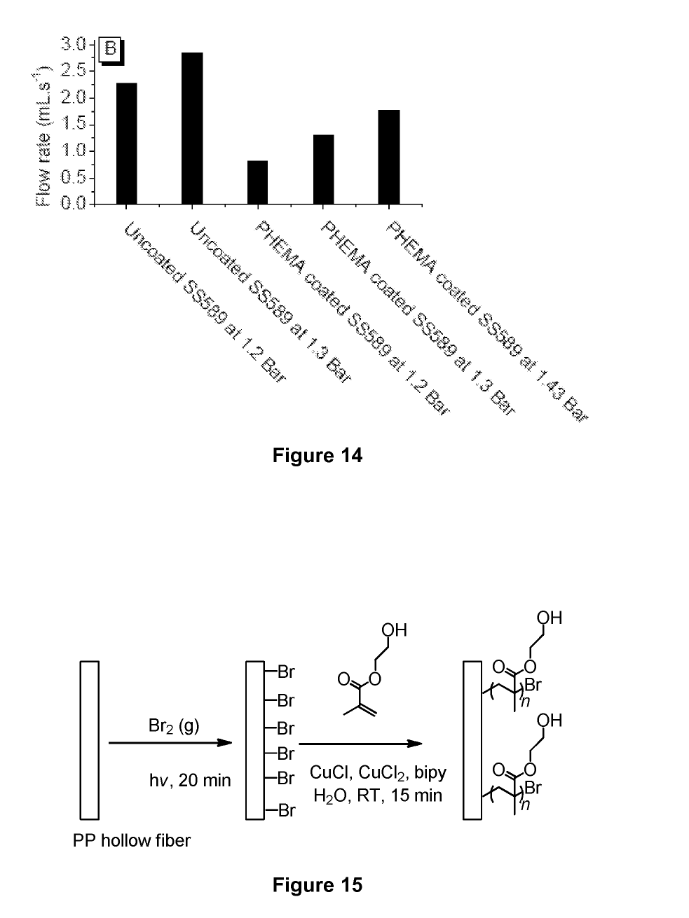

FIG. 14 shows a graphical representation of flow rate behaviour across the same membrane at different pressures (1.2 bar, 1.3 bar and 1.43 bar).

FIG. 15 shows a reaction scheme for the formation of a PHEMA polymer brush coating on the surface of a substrate, according to a further embodiment of the present invention.

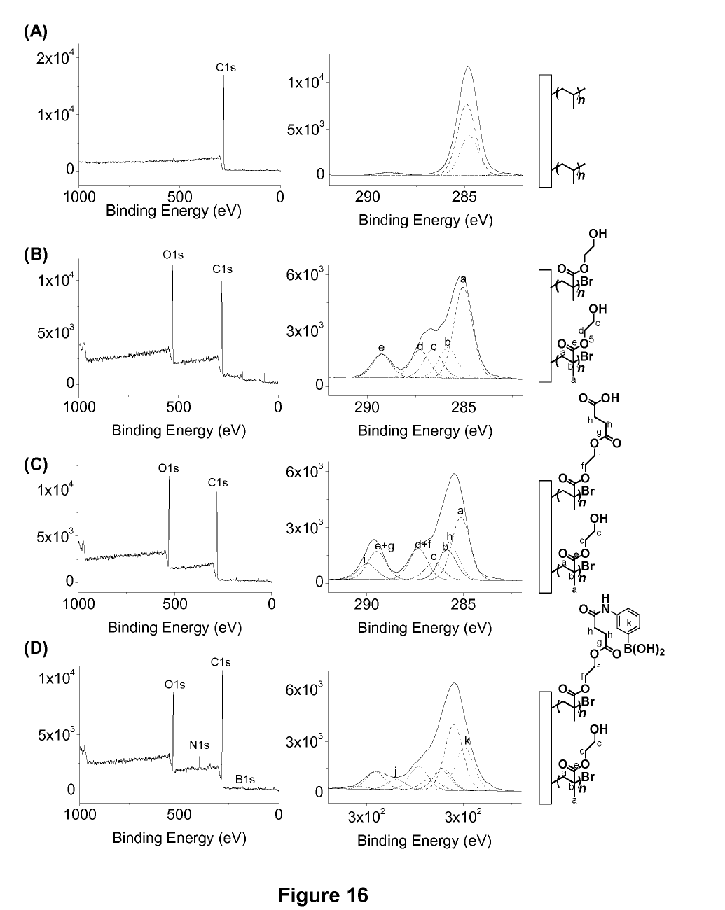

FIG. 16 shows XPS survey spectra (left) and XPS C1s (carbon) core-level spectra (right) of a membrane according to an embodiment of the invention: (A) unmodified PP hollow fiber; (B) PHEMA coated PP hollow fiber (outer part of the fiber); (C) PHEMA coated PP hollow fiber functionalized with carboxylic acid moieties; (D) PHEMA coated PP hollow fiber functionalized with phenylboronic acid moieties.

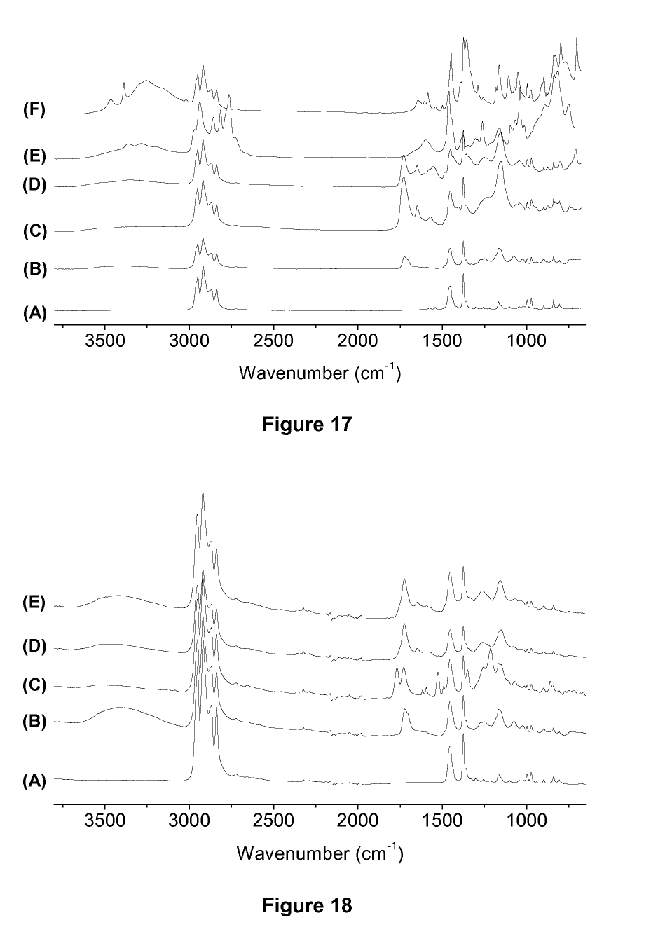

FIG. 17 shows ATR-FTIR spectra of a membrane according to an embodiment of the invention: (A) unmodified PP hollow fiber; (B) PHEMA coated PP hollow fiber; (C) PHEMA coated PP hollow fiber functionalized with carboxylic acid moieties; (D) PHEMA coated PP hollow fiber functionalized with phenylboronic acid moieties; (E) unmodified PP hollow fiber coated with 3-aminophenylboronic acid ; (F) unmodified PP hollow fiber coated with (3-(dimethylamino)-1-propylamine.

FIG. 18 shows ATR-FTIR spectra of a membrane according to an embodiment of the invention: (A) unmodified PP hollow fiber; (B) PHEMA coated PP hollow fiber; (C) PHEMA coated PP hollow fiber functionalized with NPC moieties; (D) PHEMA coated PP hollow fiber functionalized with phenylboronic acid moieties; (E) PHEMA coated PP hollow fiber functionalized with phenylboronic acid moieties and quenched with (3-(dimethylamino)-1-propylamine.

The nanoporous support substrate for the membrane may consist of any suitable nanoporous material. Examples of commercially available nanoporous substrate materials include, for example metal oxides, silica, or polymeric substrates such as polyethylene, polypropylene, polyvinylidene difluoride (PVDF), polycarbonate, cellulose (regenerated cellulose, cellulose acetate, cellulose nitrate and cellulose ester), polyethersulfone (PES), nylon, Teflon® (PTFE). Particularly nanoporous alumina membrane substrates or nanoporous polymeric substrates such as nanoporous polypropylene or polyethylene substrates may be considered.

The pore size of the pores of the nanoporous substrate may generally vary between 2 and 800 nm, preferably pore size is not more than 400 nm. At a substrate pore size of above 400 nm the control of hydraulic permeability properties of the membrane support substrate with the glucose responsive hydrogel coating layer tends to become less effective. A pore size of between 20 nm and 300 nm, e.g. between 50 nm and 200 nm, may be preferred.

A possible nanoporous substrate is nanoporous cellulose. Nanoporous cellulose membranes, e.g. re-generated cellulose, cellulose acetate or cellulose ester, are commercially available with pore sizes varying generally between 2 nm and 1000 nm. Porous hollow fibres made of cellulose, with pore size varying generally between 2 nm and 15 nm are also commercially available. Nanoporous cellulose substrates are potentially suitable for use in transcutaneous analyte sensor and regulation devices.

One type of preferred substrate materials are inert polymers such as polypropylene. Polypropylene and polyethylene substrates have found acceptance for a wide range of bio-medical applications. Compared to cellulosic materials polypropylene and polyethylene have a better durability, are more stable regarding hydrolysis, and are tolerant to aggressive chemicals which allows for a wide range of chemical modifications. Polypropylene and polyethylene nanoporous membranes with pore sizes varying generally between 20 nm and 500 nm are commercially available. Porous hollow fibres made of polypropylene and polyethylene are also commercially available, with internal diameter generally in the order of 400 μm to 2000 μm, and pore size varying generally between 100 nm and 300 nm. Particularly nanoporous polypropylene hollow fibres are of interest for application in needle-type insertion members of a medical device.

Another preferred substrate material is nanoporous alumina, e.g. produced by an anodization process. Alumina substrates have found acceptance for a wide range of bio-medical applications. Advantageously such nanoporous alumina substrates present a high porosity and a relatively uniform pore structure having substantially straight cylindrical pores. Commercially available nanoporous alumina membranes produced by anodic oxidization processes have a pore diameter dependent on, among other parameters, the applied voltage, varying generally between 5 and 200 nm. Pore size of from about 20 nm to about 200 nm may be preferred, e.g. from about 50 nm to about 150 nm.

The glucose responsive hydrogel as described herein may encompass any suitable known glucose responsive hydrogel which exhibits a reversible change in 3D configuration subject to glucose concentration in the surrounding medium. Suitable glucose responsive hydrogels include glucose responsive hydrogels having a specific affinity for glucose, which exhibit selectivity for glucose over other moieties present in physiological fluids, such as other sugars (e.g. fructose, galactose), which are sensitive to glucose at physiological conditions (temperature, ionic strength and pH values), and can respond reversibly to high and low glucose concentrations repeatably and reproducibly over many cycles (preferably over hundreds or even thousands of cycles).

The glucose responsive hydrogel should exhibit resistance to hydraulic pressure.

Advantageously the glucose responsive hydrogels according to the present invention are phenylboronic acid based hydrogels, such as described above.

Generally the hydrogels may include polymeric matrix of suitable monomer groups, such as methacrylate, acrylate, methacrylamide, acrylamide or vinylic monomer groups, functionalised with the glucose binding moiety, e.g. phenyl boronic acid moieties. The hydrogel may include cross-linker moieties, such as ethylene glycol dimethacrylate, poly(ethylene glycol) dimethacrylate, N,N′ methylenebisacrylamide, ethylene dimethacrylate, to promote hydrogel structural integrity, and/or polymer matrix density.

Particularly preferred are glucose responsive hydrogels based on phenylboronic acid derivatives. Glucose responsive hydrogels based on phenylboronic acid derivatives show good properties for resistance to flux of molecules such as water and insulin. Glucose responsive hydrogels have been developed which exhibit good selectivity for glucose, are sensitive to glucose under physiological conditions, show significant glucose response, and respond reversibly and reproducibly to high and low glucose concentrations.

Further, advantageously glucose responsive hydrogels based on phenylboronic acid or derivatives are highly stable and are resistant to heat, they can therefore be easily sterilized, e.g. by autoclave, or gamma radiation. A further advantage of the use of phenylboronic acid based hydrogels over glucose responsive hydrogels containing proteins such as glucose oxidase or lectins is that problems due to leakage of the of the proteins from the gel can be avoided.

The swelling and/or collapse of glucose responsive hydrogels based on phenyl boronic acid, or a derivative thereof, depends on the competitive binding of phenyl boronic acid moieties in the hydrogel matrix with free glucose, or on the change of solubility of the polymer in water (in the case of a temperature sensitive polymer matrix functionalised with PBA moieties), dependant on glucose concentration in the surrounding medium.

The phenylboronic acid moieties may be protected or unprotected. Particular examples of possible phenyl boronic acid based hydrogels include those described for instance in G. J. Worsley, G. A. Tourniaire, K. E. S. Medlock, F. K. Sartain, H. E. Harmer, M. Thatcher, A. M. Horgan, J. Pritchard Clinical Chem. 2007, 53, 1820-1826, Yang X P, Lee M C, Sartain F, Pan X H, Lowe C R: Designed boronate ligands for glucose-selective holographic sensors. Chemistry-a European Journal 2006, 12, 8491-8497, Kikuchi A, Suzuki K, Okabayashi O, Hoshino H, Kataoka K, Sakurai Y, Okano T: Glucose-sensing electrode coated with polymer complex gel containing phenylboronic acid. Analytical Chemistry 1996, 68:823-828, WO 2004/081624, and as discussed above. In the case of the use of monomers functionalized with protected phenylboronic acid moieties for the preparation of the glucose sensitive coating, a deprotection step is required.

Preferred phenylboronic acid moieties include the unprotected phenyl boronic acid derivatives of the structural formula (I):

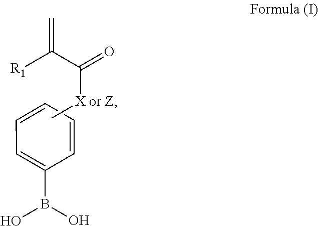

wherein X═NH or O; R₁═H or a C₁ to O₄ alkyl group; Z=a linker group. Preferably R₁=H or a methyl group. The linker group Z may be any suitable linker group such as glycol or a C₁ to C₁₀ aliphatic chain or aromatic chain. Preferred aliphatic chain groups include straight chain or branched C₁ to C₁₀ alkyl or C₁ to C₁₀ alkene groups.
or formula (II):

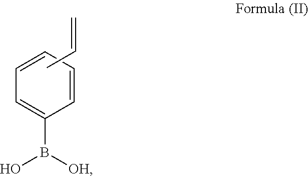

and the protected pheylboronic acid derivatives of the structural formula (III):

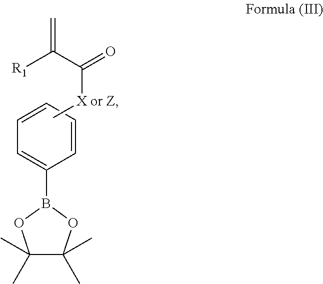

wherein X, R₁ and Z are as defined above, or (IV):

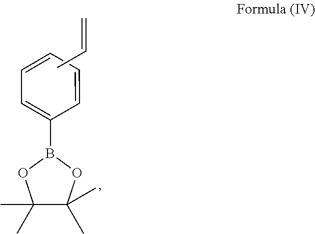

According to one embodiment preferred phenylboronic acid based hydrogels comprise a derivative of phenylboronic acid and a basic group such as a tertiary or quaternary amino group in the vicinity of the phenylboronic acid moieties, and/or a derivative phenylboronic acid modified with a tertiary or quaternary amino group.

Exemplary tertiary amino groups include a group of structural formula (V):

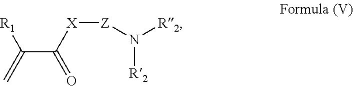

wherein X, R₁ and Z are as defined above, and R₂′ and R₂″ are each individually a C₁ to C₁₀ alkyl group, preferably a C₁ to C₄ alkyl group. R₂′ and R₂″ on a same nitrogen group may be the same or different.

Exemplary quaternary amino groups include a group of structural formula (VI):

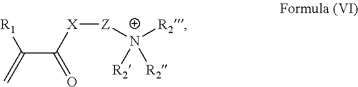

wherein X, R₁ and Z are as defined above, and R₂′, R₂″ and R₂′″ are each individually a C₁ to C₁₀ alkyl group, preferably a C₁ to C₄ alkyl group. R₂′ R₂″ and R₂ on a same nitrogen group may be the same or different.

Exemplary glucose responsive hydrogels are phenylboronic acid hydrogels comprising 3-(acrylamido)phenylboronic acid) or 2-(acrylamido)phenylboronic acid as the phenylboronic acid moiety. Particularly a phenylboronic acid hydrogel comprising 3-(acrylamido)phenylboronic acid) and a tertiary amine, such as (N-[3-(dimethylamino)propyl moiety), for instance as disclosed by G. J. Worsley, G. A. Tourniaire, K. E. S. Medlock, F. K. Sartain, H. E. Harmer, M. Thatcher, A. M. Horgan, J. Pritchard Clinical Chem. 2007, 53, 1820-1826; or a phenylboronic acid hydrogel comprising 2-(acrylamido)phenylboronic acid, for instance as disclosed by Yang X P, Lee M C, Sartain F, Pan X H, Lowe C R: Designed boronate ligands for glucose-selective holographic sensors. Chemistry-a European Journal 2006, 12:8491-8497, may be preferred.

For clinical applications, e.g. in a glucose sensor device, the glucose responsive membrane should be biocompatible in order to prevent inflammation (acute and chronic) and fibrous encapsulation of the membrane which leads to a loss of sensibility of the sensor. The hydrogel should be non-toxic and non-immunogenic. According to a preferred embodiment of the present invention the membrane further comprises anti-fouling groups. Examples of suitable bioinert groups for providing improved anti-biofouling properties include neutral groups such as polyethylene glycol (PEG), 2-hydroxyethyl and saccharide moieties, and charged groups such as phosphorylcholine moieties. PEG moieties are preferred due to the acceptance of PEG for pharmaceutical applications.

The anti-fouling groups may be introduced into the matrix of the glucose responsive hydrogel, for instance by copolymerisation of monomers functionalised with the anti-fouling groups. Advantageously the anti-fouling groups may be provided in a polymer layer attached covalently to the glucose responsive hydrogel layer, e.g. by a subsequent polymerisation process of monomers functionalised with anti-fouling groups, to form a block-copolymer.

Advantageously hydrogels according to the present invention show significant response to changes in glucose concentration, showing reversible and reproducible swelling properties subject to changes in glucose concentration. Hydrogels are able to provide good resistance to flow of water, and molecules such as insulin, due to their particular cross-linked matrix structure.

Advantageously the hydrogel is attached to a least part of the surface of the internal walls of the pores of the nanoporous substrate. An important advantage of the presence of the hydrogel on the pore walls is a synergy between the useful properties of the support substrate, such as stiffness, and those of the functional polymer layer.

The attachment of the polymers to the substrate surface may be achieved by “grafting to” techniques which involve the tethering of pre-formed functionalized polymer chains (alternatively referred to as polymer brushes) to a substrate under appropriate conditions, or “grafting from” techniques which involve covalently immobilizing an initiator species on the substrate surface, followed by a polymerization reaction to generate the polymer brushes. Creation of the polymer brushes by covalent attachment methods advantageously provides good hydrogel integrity and long-term membrane stability properties.

For the synthesis of macromolecular layers via the “grafting from” route, conventional free radical polymerization initiated by electron beam irradiation, plasma treatment, or direct UV irradiation have been used extensively. An attractive alternative to conventional UV or plasma initiated polymerization is the use of surface-initiated controlled/“living” polymerization methods such as surface initiated atom transfer radical polymerisation (SI-ATRP), nitroxide-mediated processes (SI-NMP), reversible addition-fragmentation chain transfer polymerization (SI-RAFT), photo iniferter mediated polymerization (SI-PIMP) processes and ring-opening polymerisation (SI-ROP) processes. Advantages of these controlled/“living” surface initiated polymerisation processes include precise control over the thickness and composition of the resulting polymer film, and the ability to prepare block co-polymers by sequential activation of a dormant chain end in the presence of different monomers. As polymer chains start to grow from defined initiator sites at the surface, these techniques produce thin films in which polymer chains (also referred to as polymer brushes) are tethered by their ends to the surface.

In a preferred embodiment, the glucose responsive hydrogel is created at the substrate surface by a controlled/“living” surface initiated polymerisation (SIP) process. A surface initiated controlled/“living” polymerisation process of particular interest is surface-initiated atom transfer radical polymerisation (SI-ATRP), due to its robustness and synthetic flexibility. Further, water may be used as the main solvent used for most SI-ATRP processes, which is a particular advantage for application on an industrial scale. The use of a controlled/“living” surface initiated polymerisation process for the creation of the hydrogel coating on the nanoporous substrate surface advantageously enables precise control over the thickness, polymer chain density, and composition of the hydrogel coating.

For instance, surface initiated atom transfer radical polymerisation of monomers to form a polymer coating layer on the substrate surface can be carried out in accordance with known techniques, for example, such as described in a) Zhao, B.; Brittain, W. J. J. Am. Chem. Soc. 1999, 121, 3557-3558, b) Hussemann, M.; Malmstrom, E.; McNamara, M.; Mate, M.; Mecerreyes, D.; Benoit, D. G.; Hedrick, J. L.; Mansky, P.; Huang, E.; Russel, T. P.; Hawker, C. J. Macromolecules 1999, 32, 1424-1431, c) Matyjaszewski, K.; Miller, P. J.; Shukla, N.; Immaraporn, B.; Gelman, A.; Luokala, B. B.; Siclovan, T. M.; Kickelbick, G.; Valiant, T.; Hoffman, H.; Pakula, T. Macromolecules 1999, 32, 8716-8724, d) Ejaz, M.; Yamamoto, S.; Ohno, K.; Tsuji, Y.; Fukuda, T. Macromolecules 1998, 31, 5934-5926 for silicon wafers, such as described in von Werne, T.; Patten, T. E. J. Am. Chem. Soc. 1999, 121, 7409-7410 for silica, such as described in Sun, L.; Baker, G. L.; Bruening, M. L. Macromolecules 2005, 38, 2307-2314 for alumina, such as described in a) Shah, R. R.; Merreceyes, D.; Husemann, M.; Rees, I.; Abbott, N. L.; Hawker, C. J.; Hedrick, J. L. Macromolecules 2000, 33, 597-605, b) Kim, J.-B.; Bruening, M. L.; Baker, G. L. J. Am. Chem. Soc. 2000, 122, 7616-7617, c) Huang, W.; Kim, J.-B.; Bruening, M. L.; Baker, G. L. Macromolecules 2002, 35, 1175-1179 for gold, such as described in a) Fan, X.; Lin, L.; Dalsin, J. L.; Messersmith, P. B. J. Am. Chem. Soc. 2005, 127, 15843-15847, b) Zhang, F.; Xu, F. J.; Kang, E. T. Neoh, K. G. Ind. Eng. Chem. Res. 2006, 45, 3067-3073 for titanium oxide and such as described in Li, G.; Fan, J.; Jiang, R.; Gao, Y. Chem. Mater. 2004, 16, 1835-1837 for iron oxide, and as described in, for instance, U.S. Pat. No. 6,949,292, U.S. Pat. No. 6,946,164 and WO 98/01480. Other examples are described in the following review article: Barbey, R.; Lavanant, L.; Paripovic, D.; Schuwer, N.; Sugnaux, C.; Tugulu, S.; Klok, H.-A. Chem. Rev. 2009, 109, 5437-5527.

In general, in controlled surface initiated polymerisation (SIP) processes, an initiator moiety is first chemically bound to the substrate surface, and then the polymerisation is carried out from the initiator moiety in the presence of an appropriate catalytic system. The initiating moiety generally consists of an anchoring group covalently attached to an initiating group, wherein the anchoring group is adapted to the material from which the support membrane is made of whereas the initiating group is adapted to the selected controlled SIP technique.

Dependent on the nature of the substrate material the substrate may exhibit chemical properties at its surface suitable for the binding of the initiator groups, e.g. having reactive groups, such as hydroxide groups, on their surface which act as the anchoring group for binding of the initiator group. Reactive groups may also be introduced onto the surface of the substrate by exposure to chemicals, coroner discharge, plasma treatment, etc. For example, piranha solution or plasma treatment can be used to hydroxylate, or activate, the surface of a silica or alumina substrate.

The initiator group is covalently bound to the substrate surface via the anchoring group at the substrate surface. The initiator group may be selected from known initiator groups. The choice of the initiator group for the controlled surface initiated polymerisation depends largely on the desired reaction conditions and the monomer(s) to be polymerised. Examples of suitable initiator species may be found, for example, in U.S. Pat. No. 6,949,292, U.S. Pat. No. 6,986,164, U.S. Pat. No. 6,653,415 and US2006/0009550A1.

The initiator groups may be assembled onto the surface of the substrate in the presence of appropriate solvents.

Starting from the initiator moiety bound to the substrate surface a “living”/controlled surface initiated polymerisation is carried out with the monomers as desired for the formation of the polymer brushes. The “living”/controlled free radical polymerisation reaction is carried out in the presence of a suitable catalytic system. Typical catalytic systems comprise metal complexes containing transition metals e.g. copper, ruthenium or iron as the central metal atom. Exemplary metal catalysts include copper complexes such as copper chloride, copper bromides, copper oxides, copper iodides, copper acetates, copper perchlorate, etc.

In a particular embodiment, the glucose responsive hydrogel coating may be formed by SI-ATRP from a nanoporous alumina substrate. An initiator species, for instance as described in U.S. Pat. No. 6,653,415, for example a bromoisobutyramido trimethoxysilane initiator group, or a chlorodimethylsilyl 2-bromo-2-methylpropanoate group, may be used. Other suitable initiator species include a cathecolic alkyl halide initiator group, such as 2-Bromo-N-[2-(3,4-dihydroxy-phenyl)-ethyl]-propionamide, as described in US 2006/0009550.

In the case of a nanoporous cellulose substrate SIP may be carried out from the substrate surface using a suitable initiator capable of binding with hydroxyl groups on the cellulose substrate surface. For instance the cellulose hydroxyl groups may be esterified with 2-bromoisobutyryl bromide, or analogs thereof (Carlmark, A.; Malmstrom, E. J. Am. Chem. Soc. 2002, 124, 900-901). To enhance the accessibility of the hydroxyl groups and to facilitate higher degree of substitution, cellulose fibers may, for example, be pretreated with aqueous NaOH. After extensive washing with ethanol and tetrahydrofuran (THF), these substrates may, for example, be reacted with 2-chloro-2-phenylacetyl chloride and subsequently treated with phenyl magnesium chloride in the presence of carbon disulfide to generate a cellulose-bound RAFT agent (Roy, D.; Knapp, J. S.; Guthrie, J. T.; Perrier, S. Biomacromolecules 2008, 9, 91-99)

Polymeric substrates such as polyethylene (PE) or polypropylene (PP) that lack functional groups, which can act as handles to introduce functional groups that initiate or mediate SI-CRP, generally require a pretreatment or activation step. A variety of plasma and oxidative surface treatments are known for modifying inert polymer substrates with hydroxyl or carboxylic acid groups, which can then be further modified with initiator species, such as 2-bromoisobutyryl bromide or analogues, to allow SI-ATRP. For instance, ATRP initiating groups have been introduced onto the surface of polypropylene hollow fiber membranes using ozone pretreatment (Yao, F.; Fu, G.-D.; Zhao, J. P.; Kang, E.-T.; Neoh, K. G. J. Membr. Sci. 2008, 319, 149-157) and onto the surface of high-density polyethylene (HDPE) film using maleic anhydride activation (Yamamoto, K.; Miwa, Y.; Tanaka, H.; Sakaguchi, M.; Shimada, S. J. Polym. Sci., Part A: Polym. Chem. 2002, 40, 3350-3359).

Alternatively several protocols have been developed that allow the modification of “inert” polymer substrates with SI-CRP active functional groups in a single step. For example, polypropylene (Desai, S. M.; Solanky, S. S.; Mandale, A. B.; Rathore, K.; Singh, R. P. Polymer 2003, 44, 7645-7649) and polyethylene (Lavanant, L.; Pullin, B.; Hubbell, J. A.; Klok, H.-A. Macromol. Bioscience 2010, 10, 101-108) can be photobrominated to generate alkyl bromide groups that can be used directly to initiate SI-ATRP. Another approach that allows the one step modification of “inert” polymer substrates is based on benzophenone photochemistry. Under UV radiation, benzophenone can abstract a hydrogen atom from neighboring aliphatic C—H groups to form a C—C bond. For example, the benzophenone group in benzophenonyl 2-bromoisobutyrate may be used as an anchor to promote the immobilization of the ATRP initiator group on polypropylene (Huang, J. Y.; Murata, H.; Koepsel, R. R.; Russell, A. J.; Matyjaszewski, K. Biomacromolecules 2007, 8, 1396-1399). Alternatively, benzophenone may be grafted onto high-density polyethylene (HDPE) and used as an initiator for reverse ATRP (Desai, S. M.; Solanky, S. S.; Mandale, A. B.; Rathore, K.; Singh, R. P. Polymer 2003, 44, 7645-7649.

The methods described above can suitably be used to prepare polypropylene and polyethylene substrates with functional groups that can initiate or mediate SI-CRP, e.g. SI-ATRP. Alternatively, polymer brushes can be grown from polymeric substrates, such as polypropylene or polyethylene, in the absence of such functional groups when the polymeric substrate is exposed to UV or y-irradiation or is plasma treated. For example y-irradiation may be used to initiate polymerization and cumyl phenyldithioacetate used as a RAFT agent for the grafting of polymer brushes from polypropylene substrates (Barner, L.; Perera, S.; Sandanayake, S.; Davis, T. P. J. Polym. Sci., Part A: Polym. Chem. 2006, 44, 857-864). Similarly, 1-phenylethyl phenyldithioacetate has been used as RAFT agent for the modification of the surface of polyethylene-co-polypropylene (PE-co-PP) sheets (Kiani, K.; Hill, D. J. T.; Rasoul, F.; Whittaker, M.; Rintoul, L. J. Polym. Sci., Part A: Polym. Chem. 2007, 45, 1074-1083). UV and y-radiation have also been used to activate polyethylene substrates and allow reverse SI-ATRP (Yamamoto, K.; Tanaka, H.; Sakaguchi, M.; Shimada, S. Polymer 2003, 44, 7661-7669).

By the use of controlled surface initiated polymerisation techniques to create the glucose responsive hydrogel coating on the nanoporous support substrate, a thin coating of the hydrogel can be produced on the surface of the nanoporous support substrate and the thickness of the hydrogel coating can be precisely controlled. The thickness of the hydrogel coating produced in a specific SIP polymerisation reaction is controlled by the kinetics of the reaction which can be controlled in particular by controlling the length of time of the polymerisation reaction, or the concentration of monomers/catalyst.

The thickness of the hydrogel coating on the nanoporous substrate required to provide the desired flow rate properties for the coated membrane will depend, amongst other things, on the pore size and structure of the nanoporous substrate, and the structure and nature of the selected hydrogel, e.g. swelling properties of the selected hydrogel. Preferably the thickness of the hydrogel coating is between 1 nm and 300 nm. For further improved responsivity of the membrane to glucose, coating thickness of between 1 nm and 200 nm, may be preferred, for instance between 1 nm and 100 nm, e.g. from 5 nm to 100 nm, for example from 5 nm to 50 nm, e.g. from 5 nm to 20 nm.

Advantageously the use of controlled surface initiated polymerisation techniques to form a glucose responsive hydrogel coating on the surface enables the preparation of a thin layer of the hydrogel which is strongly attached through covalent bonds to the surface of the nanoporous support substrate; thereby providing a glucose responsive membrane exhibiting a rapid response to changes in glucose concentration, whilst at the same time showing good hydrogel integrity and long term stability properties (e.g. over 5 to 7 days under pharmacological conditions) required for clinical applications.

A glucose responsive membrane according to the present invention may suitably be prepared by a process comprising the steps of covalently binding an initiator group to a surface of a nanoporous support substrate, and subsequently forming a glucose responsive hydrogel at the surface of the nanoporous substrate via a controlled surface initiated polymerisation process from the initiator group.

According to an embodiment of the present invention the formation of the glucose responsive hydrogel at the surface of the nanoporous substrate via a controlled surface initiated polymerisation process may involve the co-polymerisation of monomers selected from methacrylate, acrylate, methacrylamide, acrylamide or vinylic monomers functionalised with glucose binding functional groups, methacrylate, acrylate, methacrylamide, acrylamide or vinylic monomers functionalised with tertiary or quaternary amino groups, methacrylate, acrylate, methacrylamide, acrylamide or vinylic monomers functionalised with active ester groups, cross-linker groups selected from di(methacrylate), di(acrylate), di(methacrylamide), di(acrylamide) or di(vinylic) monomer groups, methacrylate, acrylate, methacrylamide, acrylamide or vinylic monomers functionalised with 2-hydroxyethyl, polyethylene glycol or phosphorylcholine groups, non-functionalised methacrylate, acrylate, methacrylamide, acrylamide or vinylic monomers.

Phenylboronic acid, or derivatives of phenylboronic acid, glucose binding functional groups may be introduced in the glucose responsive hydrogel by direct co-polymerisation of monomers comprising glucose binding functional groups in a controlled surface initiated polymerisation process, from the initiator group.

Alternatively, glucose binding functional groups may be introduced in the glucose responsive hydrogel in a subsequent step by substitution of glucose binding functional group containing moieties at activated sites in the formed hydrogel.

Advantageously the glucose responsive hydrogel comprises anti-fouling functional groups to prevent the non-specific adhesion of proteins, present in interstitial fluid, to the surface of the glucose responsive membrane.

The anti-fouling groups, such as 2-hydroethyl or polyethylene glycol (PEG), may be introduced into the matrix of the glucose responsive hydrogel, for instance by copolymerisation of monomers functionalised with the anti-fouling groups in a controlled surface initiated polymerisation process from the initiator groups bound to the substrate surface.

Advantageously the anti-fouling groups may be provided in a polymer layer attached covalently to the glucose responsive hydrogel layer, e.g. by a subsequent controlled surface initiated polymerisation step of monomers functionalised with anti-fouling groups, to form a block-copolymer. The use of a controlled surface initiated polymerisation process enables the formation of a thin layer of biocompatible polymer covalently attached to the glucose responsive hydrogel, and of which the layer thickness can be precisely controlled. The protection of the glucose responsive layer with a thin layer of covalently attached biocompatible polymer brushes, to form a block co-polymer, in this way is particularly preferred in the case of a glucose responsive hydrogel based on phenylboronic acid moieties since glycoproteins such as y-globulin, present at non negligible concentrations in interstitial fluid, are known to interact with phenylboronic acid moieties which could lead to undesirable foreign body reaction.

Examples of suitable process strategies for the preparation of a glucose sensitive hydrogel comprising phenylboronic acid moieties are illustrated schematically in FIG. 1.

According to a first possible process strategy, illustrated in FIG. 1A, in a first controlled surface initiated polymerisation step from initiator groups (3) bound to the nanoporous support substrate (1) there are copolymerised (i) methacrylate or acrylate or methacrylamide or acrylamide or vinylic monomers functionalized with unprotected phenylboronic acid moieties (5), preferably selected from unprotected phenylboronic acid moieties of formula (I) or (II), or protected phenyl boronic acid preferably selected from unprotected phenylboronic acid moieties of formula (I) or (II), and optionally,

(ii) monomers such as non-functionalized methacrylate or acrylate or methacrylamide or acrylamide or vinylic monomers, cross-linkers, and/or methacrylate or acrylate or methacrylamide or acrylamide or vinylic monomers functionalized with neutral tertiary amine groups (7), preferably selected from a group of formula (III), or charged quaternary amine groups, preferably selected from a group of formula (IV).

Optionally, methacrylate or acrylate or methacrylamide or acrylamide or vinylic monomers functionalized with anti-fouling functional groups, such as 2-hydroethyl or polyethylene glycol moieties, may be copolymerized with the above-listed monomers in the first polymerisation step or methacrylate or acrylate or methacrylamide or acrylamide or vinylic monomers functionalized with anti-fouling functional groups (9), such as 2-hydroethyl or polyethylene glycol moieties, may be polymerized in a second polymerization step to give a block-copolymer (FIG. 1D).

Suitable cross-linkers include groups of the general structural formula (VII):

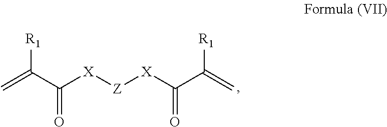

wherein X, R₁ and Z are as defined above.

According to a second possible process strategy, illustrated in FIG. 1B, in a first controlled surface initiated polymerisation step from initiator groups bound to the nanoporous support substrate there are copolymerised

(i) methacrylate or acrylate or methacrylamide or acrylamide or vinylic monomers functionalized with active ester groups (11), and optionally,
(ii) monomers such as non-functionalized methacrylate or acrylate or methacrylamide or acrylamide or vinylic monomers and/or cross-linkers, such a cross-linker groups of formula (VII).

Optionally, monomers such as non-functionalized methacrylate or acrylate or methacrylamide or acrylamide or vinylic monomers monomers functionalized with anti-fouling functional groups, such as 2-hydroethyl or polyethylene glycol moieties, may be copolymerized with the above-listed monomers in the first polymerisation step or may be polymerized in a second polymerization step to give a block-copolymer (FIG. 1D).

In a subsequent step phenylboronic acid functional groups, and optionally tertiary or quaternary amino functional groups, are introduced at the active ester sites by nucleophilic substitution.

Suitable active ester functional groups include active ester groups of the structural formulae (VIII):

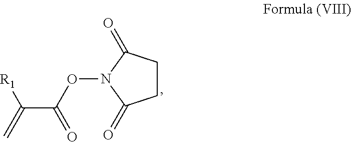

### or (IX)

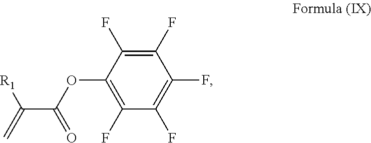

wherein R₁ is as defined above.

Phenylboronic acid functional groups may, for instance, be introduced at the active ester sites by nucleophilic substitution of a group of structural formula (X):

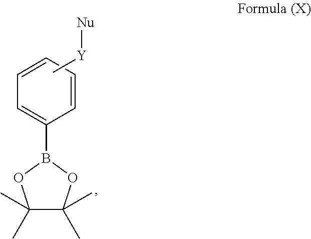

### or (XI):

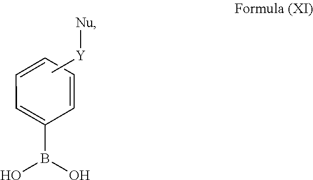

wherein Y═NH₂ or a linker group. The linker group may be any suitable linker group such as glycol or a C₁ to C₁₀ aliphatic chain or aromatic groups. Preferred aliphatic chain groups include straight chain or branched C₁ to C₁₀ alkyl or C₁ to C₁₀ alkene groups. Nu=a nucleophillic group. Suitable nucleophillic groups include NH₂ or OH.

Amino functional groups may, for instance, be introduced at the active ester sites by nucleophilic substitution of a group of structural formula (XII):

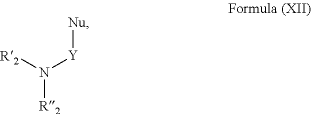

wherein Y, Nu, R₂′ and R₂″ are as defined above.

According to a third possible process strategy, illustrated in FIG. 1C, in a first controlled surface initiated polymerisation step from initiator groups bound to the nanoporous support substrate there are copolymerised

(iii) methacrylate or acrylate or methacrylamide or acrylamide or vinylic monomers functionalized with anti-fouling functional groups (9), such as 2-hydroethyl or polyethylene glycol moieties, and optionally,
(iv) monomers such as non-functionalized methacrylate or acrylate or methacrylamide or acrylamide or vinylic monomers and/or cross-linkers, such a cross-linker groups of formula (VII).

In a subsequent step anti-fouling functional groups (9), e.g. 2-hydroethyl or polyethylene glycol moieties, are modified with active esters (13), such as active ester groups of formula (VIII) or (IX).

In a subsequent step phenylboronic acid functional groups, and optionally tertiary or quaternary amino functional groups, are introduced at the active ester sites by nucleophilic substitution. Phenylboronic acid functional groups may, for instance, be introduced at the active ester sites by nucleophilic substitution of groups of structural formula (X) or (XI). Amino functional groups may, for instance, be introduced at the active ester sites by nucleophilic substitution of groups of structural formula (XII).

The strategies A and B can be combined. For example, in a first controlled surface initiated polymerisation step from initiator groups bound to the nanoporous support substrate there may be copolymerised

(i) methacrylate or acrylate or methacrylamide or acrylamide or vinylic monomers functionalized with neutral tertiary amine groups, preferably selected from a group of formula (III), or charged quaternary amine groups, preferably selected from a group of formula (IV),
(ii) methacrylate or acrylate or methacrylamide or acrylamide or vinylic monomers functionalized with active ester groups, such as active ester groups according to formula (VIII) or (IX), and optionally (iii) non-functionalized methacrylate or acrylate or methacrylamide or acrylamide or vinylic monomers, cross-linkers, such a cross-linker groups of formula (VII).

Optionally, methacrylate or acrylate or methacrylamide or acrylamide or vinylic monomers functionalized with anti-fouling functional groups, such as 2-hydroethyl or polyethylene glycol moieties, may be copolymerized with the above-listed monomers in the first polymerisation step or may be polymerized in a second polymerization step to give a block-copolymer (FIG. 1D).

In a subsequent step phenylboronic acid functional groups, are introduced at the active ester sites by nucleophilic substitution. The phenylboronic acid functional groups may, for instance, be introduced at the active ester sites by nucleophilic substitution of groups of structural formula (X) or (XI).

The glucose responsive hydrogel coating layer formed according to the present invention has the effect of controlling the hydraulic flow properties of the nanoporous support substrate.

As discussed above the degree of swelling and/or collapse of hydrogel polymer matrices functionalised with phenyl boronic acid, or a derivative thereof, depends on glucose concentration in the surrounding medium. In the glucose responsive hydrogel coated membrane the degree of opening of the pores of the nanoporous support substrate, that is to say the size of open flow channel through the pores, is controlled by changes in the degree of swelling of the glucose responsive hydrogel, subject to changes in glucose concentration in the surrounding medium.

Accordingly, the degree of opening of the pores of the membrane substrate, conveniently measured as an average diameter of the pores, changes as a function of glucose concentration in the surrounding medium.

Another important property of phenylboronic acid compounds in an aqueous medium is that they are in equilibrium between an uncharged and a charged form. Only charged phenylborates can form stable complex with glucose.

Increasing glucose concentration increases the charged phenylborates, thus, enhancing the hydrophilicity of amphiphilic polymers having pendant phenylborate moieties (see J. Xingju, Z. Xinge, W. Zhongming, T. Dayong, Z. Xuejiao, W. Yanxia, W. Zhen, L. Chaoxing Biomacromolecules 2009, 10, 1337-1345).

It is considered by the inventors that the variation of hydrophilicity of the hydrogel in response to glucose concentration could influence the surface energy of the material coated with PBA-based hydrogel. Indeed, it has been demonstrated that the surface energy of capillaries in microfluidic systems plays a crucial role in flow behavior (see L. Ionov, N. Houbenov, A. Sidorenko, M. Stamm, S. Minko Adv. Funct. Mater. 2006, 16, 1153-1160. A. Constable, W. Brittain, Colloids and Surfaces a-Physicochemical and Engineering Aspects, 2007, 308, 123-128. in addition to volume changes. Without wishing to be bound by any particular theory it is considered by the inventors that variation of hydrophilicity of the hydrogel in response to glucose concentration also plays a role in the control of the hydraulic permeability of the membrane in response to glucose concentration.

Attachment of the glucose responsive hydrogel to inner walls of pores of the nanoporous substrate provides enhanced control of flux properties through the membrane, since the swelling of the hydrogel coating present in the pore produces a restriction of open pore diameter (i.e. flow channel size) along the length of the pore. In addition where hydrogel is present in pores of the porous substrate the change in hydrophobicity of the hydrogel in response to glucose concentration may play an enhanced role in the control of hydraulic permeability of the membrane. The presence of the glucose responsive hydrogel in the pores of the nanoporous substrate promotes effective closing of the pores by the hydrogel in a swollen state, due to increased resistance to hydraulic flow through the pore.

Glucose responsive membranes of the present invention are able to provide a rapid response time to changes in glucose concentration, whilst providing good hydrogel integrity and stability properties.

Advantageously, glucose responsive membranes according to the present invention show significant response to changes in glucose concentration at physiological conditions. Advantageously, glucose responsive membranes according to the present invention can provide good selectivity for glucose, and reversible and reproducible swelling properties subject to changes in glucose concentration. Advantageously, glucose responsive membranes according to the present invention provide good resistance to flux of water, and molecules such as insulin.

Further, glucose responsive membranes of the present invention provide good bio-compatibility properties.

Advantageously glucose responsive membranes according to the present invention comprising phenylboronic acid based glucose responsive hydrogel can be easily sterilised, e.g. by autoclave or gamma radiation, as required for in-vivo clinical applications.

The glucose responsive membranes of the present are particularly advantageous for the use in medical device applications for the monitoring or regulation of glucose levels.

Particularly, the coated membranes of the present invention are advantageously used in glucose sensor device, or a medical device for the treatment of patients with diabetes, particularly a closed loop system integrating glucose sensor and medication delivery.

Advantageously the medical device for the monitoring or regulation of glucose levels is based on mechanical sensing methods. According to a preferred embodiment, the medical device determines glucose concentration in a patient body fluid based on measurement of a flow resistance of a liquid through the glucose responsive membrane.

According to one embodiment of a medical device according to the present invention, as illustrated in FIG. 2 the glucose responsive membrane of the present invention, comprising a nanoporous support substrate (20), and a glucose responsive hydrogel coating (22) may form a bio-interface of part of a needle-like insertion member (24) to be inserted in a patient to contact with e.g. interstitial fluid, blood or tear fluid. In order to measure glucose concentration in the medium surrounding the needle-like insertion member a liquid flux (26) is produced in the insertion member, and the resistance to flux of this liquid through the glucose responsive membrane is measured. The change in volume and/or surface properties of the glucose response hydrogel coating attached to the support substrate, subject to the glucose concentration in the medium surrounding the needle-like insertion member, has the effect of decreasing or increasing the resistance to flux of the liquid through the membrane. The resistance to flux of the liquid through the membrane is measured using a flux sensor device, and this value used to determine glucose concentration in the medium surrounding the needle-like insertion member.

A medication capable of regulating blood glucose levels, e.g. insulin, may be delivered by the medical device in response to the determined glucose concentration.

According to a particular embodiment the glucose responsive hydrogel (22) may comprise phenyl boronic acid moieties (28) and tertiary amine moieties (30). At low glucose concentration in the surrounding medium the glucose responsive hydrogel has an expanded configuration (32), thereby closing or narrowing the pore diameter of the nanoporous substrate. The swelling of the hydrogel coating layer and/or the change in surface properties (i.e. hydrophilicity) of the glucose response hydrogel coating have the effect of decreasing the effective cross-section of the pores of the nanoporous substrate, and this swollen hydrogel coating layer on the surface of the nanoporous substrate provides a high resistance to flux of the liquid through the membrane. At a high glucose concentration glucose (34) is bound by the phenylboronic acid moieties and contraction of the hydrogel occurs, whereby the contraction of the hydrogel coating layer and/or the change in surface properties (i.e. hydrophilicity) of the glucose response hydrogel coating have the effect of increasing the effective cross-section of the pores of the nanoporous substrate, and the contracted hydrogel coating layer (34) on the surface of the nanoporous substrate provides a lower resistance to flux of the liquid through the membrane. Advantageously, the closing of the pores of the nanoporous membrane substrate at low glucose concentration in this way may increase the sensitivity of the system in the hypoglycaemic region, which is known to be difficult to monitor accurately with electrochemical sensors.

The medical device may, for example, have a construction of medical device for glucose monitoring and for drug delivery similar to that described in PCT/IB2008/054348, and wherein the medical device comprises an implantable member, having a needle-like form, for insertion into a patient, comprising a porous membrane, a pressure generating means adapted to deliver a liquid to the porous membrane, and a sensor adapted to measure flow resistance of the liquid through the membrane. A glucose sensitive membrane according to the present invention, which changes it hydraulic permeability subject to changes in glucose concentration in the medium contacting the membrane, may be used in the place of the porous membrane described in PCT/IB2008/054348.

Glucose concentration is measured by pumping a discrete volume of liquid towards the membrane, measuring a value correlated to a resistance against flow of the liquid through the membrane, and calculating a glucose concentration based on the measured value correlated to flow resistance through the porous membrane.

The liquid may comprise insulin, such that it is possible for the device to provide e.g. a basal rate of insulin through glucose responsive membrane. The medical device may comprise a separate channel for drug delivery, such that a bolus insulin dose may be administered through this separate channel as required, according to the determined glucose concentration.

Other constructions for the glucose sensor or medication delivery device may be envisaged.

The invention may be further illustrated by the following non-limiting examples.

### EXAMPLES

### Materials

2-hydroxyethyl methacrylate (HEMA) was obtained from Aldrich and freed from the inhibitor by passing the monomer through a column of activated, basic aluminum oxide. 2,2″-bipyridine (bipy), Cu(II) chloride (99.999%), Cu(I) chloride (purum, ≧97%), dimethylaminopryridine (DMAP), succinic anhydride, N-3-dimethyl(aminopropyl)-N′-ethylcarbodiimide hydrochloride (EDAC), 3-aminophenylboronic acid (PBA, 98%), and 2-(N-morpholino)ethanesulfonic acid (MES) buffer were purchased from Aldrich and were used as received. Triethylamine (TEA) was purchased from Aldrich and was distilled over KOH. Tetrahydrofurane (THF) and dimethylformamide (DMF) were purified and dried using an automated solvent purification system (PureSolv). Deionized water was obtained from a Millipore Direct-Q 5 Ultrapure Water System. Cellulose filter grade SS589/3 (particle retention in liquid <2 μm, thickness: 160 μm) was purchased from Whatman. The PP hollow fibers MICRODYN® (MD 070 FP 1 L, inner diameter: ˜600 μm, pore size: ˜100 nm) were sourced from Microdyn Nadir. Anodic aluminium oxide (AAO) membranes (Whatman®, Anodisc 25) with pore diameter of 0.2 μm, average thickness of 60 μm and supported by a polypropylene ring were purchased from Whatman. The membranes were used as received without cleaning step.

### Example 1

### Synthesis of PHEMA-Coated AAO Membranes and Post-Functionalization with PBA Moieties

Synthesis of the PHEMA polymer brush coating was carried out from AAO membranes according the reaction scheme illustrated in FIG. 3. The post-functionalization of the PHEMA brushes with PBA groups was carried out according to the reaction scheme illustrated in FIG. 4.

Step 1: Immobilization of ATRP Initiators onto the Surface of the AAO Membranes.

The ATRP initiator 5-(2-bromo-2-methylpropanamido)-2-hydroxybenzoic acid) was prepared as described in patent application US 2009/112075. The nanoporous alumina membranes were incubated overnight at pH=5 and ionic force I=0.01 mM (NaCl). After incubation, the membranes were immersed in a 5 mM solution of the ATRP initiator (the initiator is first dissolved in 100 μl of acetone) in water (pH=5 and I=0.01 mM). The reaction was allowed to process overnight. The initiation solution was then removed; the membranes were rinsed thoroughly with water, dried under a stream of nitrogen and were used immediately.

Step 2: Grafting of HEMA from the Surface of the AAO Membranes Functionalized with ATRP Initiators:

Surface-initiated atom transfer radical polymerization of HEMA was carried from the initiator coated AAO membranes following the method described in Example 1 above to form the PHEMA polymer brush coated membranes.

Step 3: Functionalization of the PHEMA Brushes with PBA Moieties:

PHEMA brushes were activated by exposure to a freshly prepared solution of p-nitrophenyl chloroformate (NPC) (282 mg, 1.4 mmol) and triethylamine (0.19 mL, 1.4 mmol) in anhydrous THF (30 mL) for 1 h at room temperature under vigorous shaking. The modified AAO membranes were extensively rinsed with anhydrous THF to remove unreacted NPC from the surfaces and then dried under a flow of nitrogen. Activated PHEMA brushes were used immediately and functionalized by treatment with a solution containing PBA (2.6 mg, 0.015 mmol), 3-(dimethylamino)-1-propylamine (1.9 μL, 0.015 mmol) and 4-(dimethylamino)pyridine (DMAP) (2 mg, 0.016 mmol) in anhydrous DMF (10 mL) overnight at room temperature under stirring in the dark. The PBA-modified brushes were then washed with DMF, rinsed with water, acetone and ethanol to remove residual unreacted PBA groups and was finally dried in a stream of nitrogen.

The modified AAO membranes were characterized by X-ray photoelectron spectroscopy (XPS). XPS was carried out using an Axis Ultra instrument from Kratos Analytical. The XPS C1s (carbon) core level spectra of the PHEMA brushes grafted from the AAO membrane is shown in FIG. 5. The C1s (carbon) core-level spectrum recorded after a polymerization time of 2 hours can be curve fitted with five peak components at 285.0, 285.8, 286.5, 287.1 and 289.1 eV respectively, which can be attributed to the aliphatic backbone (C—H), aliphatic backbone (C—H), ethylene glycol units (C—OH), ethylene glycol units (C—O—C) and to the ester groups (C═O—O) of the PHEMA chain. The introduction of the NPC groups and attachment of the PBA moieties were confirmed by XPS experiments. After conjugation with PBA, the XPS spectrum shows the appearance of a new carbonyl signal at 288.1 eV attributed to the presence of amide bond (FIG. 5) and the XPS survey spectrum shows the presence of N1s (nitrogen) and B1s (boron) signals (FIG. 5).

### Example 2

### Permeability of PBA Functionalized PHEMA-Coated AAO Membranes

The ability of the AAO membranes modified with PHEMA brushes and functionalized with PBA groups (prepared according to example 1) to respond to glucose was evaluated. A commercially available dead-end ultrafiltration (UF) stirred-cell (Amicon® model 8010 provided by Millipore) equipped with a stirring system (a magnetic stir bar rotates freely above the membrane surface) was used. The ultra-filtration cell was connected to a compressed air line which was used as a pressure source, for ensuring supply of fluid to the coated surface of the membrane under pressure. The stirred-cell has a membrane diameter of 25 mm and an effective membrane area of 4.1 cm2. The stir speed was set at 400 rpm and the pressure fixed at 1.20 bar during the measurement. The pressure was measured using a pressure sensor interfaced with a computer. An electronic balance, interfaced with a computer, was used to weigh the permeate solution flow through the membrane continuously throughout the operating time. The feed tank of the stirred-cell was refilled after each measurement using a syringe.

The influence of the ATRP time on the flow properties of the PHEMA modified AAO membranes was evaluated. For these series of experiments, a non-buffered solution at pH 6 was used. The PHEMA modified AAO membranes were first incubated 2 h in water and then, the flows were calculated on an average of five measurements of 60 seconds and reported with the corresponding standard error. The increase in the ATRP time induced a decrease in the flow rates through the membranes (FIG. 6).

In a second series of experiments, the flow rates through PHEMA grafted AAO membranes (obtained after 2 h of ATRP) and those functionalized with PBA groups were evaluated in the presence and in the absence of glucose. The membranes were incubated 2 h in a solution of glucose (6 mmol) at pH 9 (borate buffer) under slight stirring and then the flow rates were evaluated using the same buffer. The membranes were then incubated 2 h in borate buffer at pH 9.0 and flow rates were determined.

As seen from FIG. 7, the flow rates through the PHEMA grafted AAO membranes after incubation in borate buffer were similar to those measured after incubation in glucose, which indicates that these membranes are not sensitive to the presence of glucose. The flow rates through membranes functionalized with PBA groups showed lower flow rates after incubation in borate buffer when compared to the flow rates obtained after incubation in glucose, which indicates that the membranes functionalized with PBA groups are sensitive to the presence of glucose. Linear flow measurement profiles were obtained in the case of PHEMA grafted AAO membranes, incubated in glucose solution (FIG. 8B) and incubated in borate buffer (FIG. 8D), and in the case of membranes functionalized with PBA groups incubated in borate buffer (FIG. 8C), whereas a non linear profile was obtained in the case of membranes functionalized with PBA groups incubated in glucose (FIG. 8A), indicating that the membranes functionalized with PBA groups are sensitive to glucose.

### Example 3

### Synthesis of poly(2-hydroxyethyl Methacrylate) (PHEMA)-Coated Nanoporous Substrate and Post-Functionalization with PBA Moieties

Synthesis of the PHEMA polymer brush coating was carried out from a Whatman cellulose filters (SS589/3) according the reaction scheme illustrated in FIG. 9. The post-functionalization of the PHEMA brushes with PBA groups was carried out according to the reaction scheme illustrated in FIG. 10.

Step 1: Immobilization of the ATRP Initiator onto the Surface of the SS589/3 Substrate

SS589/3 substrates were washed with acetone and THF prior to use and equilibrated in dry THF for 2 h. The hydroxyl groups on the surface were then reacted by immersing the substarte in a solution containing 2-bromoisobutyrylbromide (50 mM), triethylamine (55 mM), and a catalytic amount of DMAP (1 mM) in THF. The reaction proceeded at room temperature overnight. SS589/3 substrates were thereafter thoroughly washed with THF and ethanol and slightly ultrasonicated for 30s each time in both solvents.

Step 2: Grafting of Hema from the Surface of the SS589/3 Substrates Functionalized with ATRP Initiators

SS589/3 substrates are fragile and so, separated polymerization reactors were used for each sample. The polymerization reactor consisted of a conical flask which allows stirring of the ATRP solution with a tiny magnet without damaging the membrane. Surface-initiated ATRP of HEMA was carried out using a reaction system consisting of HEMA, CuCl, CuCl₂ and bipy in the following molar ratios: 1000:8.5:1.1:23. The polymerizations were performed in water. In a typical experiment, 100 mg (0.74 mmol) of CuCl₂ and 2.440 g (15.60 mmol) of bipy were dissolved in a mixture of 80 mL of HEMA (664.00 mmol) and 80 mL of water. After degassing by three freeze-pump-thaw cycles, 550 mg (5.60 mmol) of CuCl was added. Degassing was continued for 2 cycles. The resulting solution was subsequently transferred via a cannula to the nitrogen purged reaction vessel containing the SS589/3 substrate functionalized with the ATRP initiator and the reaction was allowed to proceed at room temperature for the desired reaction time. The substrate was removed from the reactor and extensively washed with water (5 times) and ethanol (5 times) to remove residual physisorbed monomers/polymers and was finally stored in water at pH 6.

Step 3: Functionalization of the PHEMA Brushes with Carboxyl Moieties

1.25 g of succinic anhydride (12.50 mmol) was dissolved in 100 mL of dry THF and the SS589/3 substrate modified with PHEMA brushes was introduced into the solution. 1.52 g of DMAP (12.50 mmol) and 3.76 mL of TEA (27 mmol) were added to initiate the reaction. The reaction was allowed to proceed for 24 h at room temperature to produce the PHEMA chains with carboxylterminated side chains. The resulting substrate (PHEMA-COOH) was thereafter washed with copious amounts of ethanol and deionized water to remove the adsorbed reagents prior to the subsequent reaction.

Step 4. Functionalization of the PHEMA-COOH Substrate with PBA Moieties

PBA functional groups were incorporated into the brush using EDAC as a zero-length crosslinker to conjugate PBA to the PHEMA brushes functionalized with carboxyl groups. The SS589/3 substrates were immersed in MES buffer (pH 4.8, 20 mM ionic strength) and a solution of PBA (0.16 mmol) in MES buffer was added. After 15 min, a freshly prepared solution of EDAC (0.17 mmol) in MES buffer was added and the reaction was allowed to proceed overnight. The substrates were washed with copious amounts of MES buffer and then distilled water.

The modified SS589/3 substrates were characterized by X-ray photoelectron spectroscopy (XPS) and attenuated total reflectance Fourrier transform infrared (ATR-FTIR) spectroscopy. ATR-FTIR spectroscopy was carried out on a Nicolet Magna-IR 560 spectrometer equipped with a Specac Golden Gate single reflection diamond ATR system. Each spectrum was collected by accumulating 128 scans at a resolution of 4 cm⁻¹. XPS was carried out as in Example 1. The XPS C1s (carbon) core level spectra of the PHEMA brushes grafted from the SS589/3 substrates recorded after a polymerization time of 2 hours were curve fitted with five peak components at 285.0, 285.8, 286.5, 287.1 and 289.1 eV respectively, which can be attributed to the aliphatic backbone (C—H), aliphatic backbone (C—H), ethylene glycol units (C—OH), ethylene glycol units (C—O—C) and to the ester groups (C═O—O) of the PHEMA chain. The grafting of the PHEMA brushes from SS589/3 substrates can be conveniently monitored using ATR-FTIR spectroscopy (FIG. 11). FIG. 11A shows the ATR-FTIR spectra of the unmodified SS589/3 substrate. The ATR-FTIR spectra show bands at 1728, 1274, 1251 and 1135 cm⁻¹, attributed to C═O, C—O (ester), C—O (alcohol) and C—O (ether) stretching vibrations respectively (FIG. 11B).

PBA moieties were introduced onto the PHEMA modified SS589/3 substrate as shown in FIG. 10 in a two steps strategy that involves the introduction of carbonyl groups and subsequent reaction with PBA. The introduction of the carboxyl groups and attachment of the PBA moieties were confirmed by ATR-FTIR and XPS experiments. The XPS C1s (carbon) core level spectrum of the PHEMA brushes with carboxyl terminated side chains showed two new peaks appear at 289.5 and 285.6 eV, which are attributed to the aliphatic backbone (C—H) and ester groups (C═O—O) of the oxobutanoic acid moieties. The intensity of the carbonyl band at 1728 cm⁻¹ dramatically increases after the introduction of the oxobutanoic acid moieties (FIG. 11C). After conjugation with PBA, the XPS spectrum shows the appearance of a new carbonyl signal at 288.1 eV attributed to the presence of amide bond and the XPS survey spectrum shows the presence of N1s (nitrogen) and B1s (boron) signals (FIG. 12D). The FTIR-ATR spectrum of the PBA functionalized PHEMA brushes shows the appearance of a new band at 1650 cm⁻¹ which confirms the presence of the amide bond (FIG. 11D).

### Example 4

### Permeability of PHEMA-Coated Nanoporous Substrate

The permeability of the SS589/3 substrates modified with PHEMA brushes (prepared according to example 3) was evaluated. A commercially available glass filtration funnel was modified and used to investigate the water flow properties of the modified SS589/3 substrates. The flow measurement cell consisted of a glass filtration funnel with a volume capacity of 250 mL and an inner diameter of 55 mm, closed with a cap and connected to N₂ tank. After the membrane was fixed, the solution reservoir was filled with water and the system was pressurized to the operating pressure of 1.1 to 1.3 Bar. The volume of permeated water was monitored as a function of the time.

The resultant flow measurement curves at pH 6 of the uncoated glass filter (□); unmodified SS589/3 substrate (∘); SS589/3 substrate coated with PHEMA brushes obtained after 45 min of ATRP(⋄); and SS589/3 substrate coated with PHEMA brushes obtained after 180 min of ATRP (Δ), under 1.2 bar of pressure, are shown in FIG. 13(A).

As seen from FIG. 13A, the volume of permeated water increases linearly with time and slots were used to determine flow rates (FIG. 13B). The influences of the ATRP time and of the operating pressure on flows were evaluated. For these series of experiments, a non-buffered solution at pH 6 was used. The increase in the ATRP time induced a linear decrease in the flow rates through the PHEMA grafted SS589/3 substrates (FIG. 13B). As expected the increase in operating pressure induced a linear increase in the flow rates (FIG. 14).

Glucose sensitivity of the PBA functionalized PHEMA polymer brush coating, has been demonstrated above (Example 2, FIGS. 7, 8).

### Example 5

### Stability of PHEMA Brush Coating

The stability of the PHEMA polymer brush coating prepared according to example 3 was evaluated. After the series of flow measurements presented in example 4, the unmodified and modified SS589/3 substrates were washed with buffer at pH9 and incubated overnight in the same buffer. The unmodified and modified SS589/3 substrates were then analyzed by XPS and ATR-FTIR spectroscopy (carried as in example 1). No modification of the chemical composition of the unmodified or modified SS589/3 substrates was observed which indicates that PHEMA brushes are strongly attached to cellulose substrate and no hydrolysis of the PHEMA backbone occurred during the flow measurements.

### Example 6

### Synthesis of PHEMA-Coated Polypropylene (PP) Hollow Fibers and Post-Functionalization with PBA Moieties

Synthesis of the PHEMA polymer brush coating was carried out from a polypropylene hollow fiber according the reaction scheme illustrated in FIG. 15. The post-functionalization of the PHEMA brushes with PBA groups was carried out according to the reaction schemes illustrated in FIGS. 10 and 4.

### Step 1: Photobromination of the Polypropylene Hollow Fibers.

A piece of hollow fiber membrane (60 mm length) was introduced in a 5×150 mm (diameter×length) Pyrex glass tube, which was subsequently sealed with a septum. After that, the flask was purged with nitrogen for 60 min and 10 μL of bromine was introduced with a syringe. After 5 min, when the bromine had vaporized, the tube was placed in front of a Hamamatsu LC6 high-pressure vapor mercury lamp (HPMV), which was equipped with a condenser lens in order to obtain a uniform illumination of the film. The lamp was operating at 100% intensity and placed at a distance of 33 cm from the Pyrex tube to generate a spot with a diameter of 12 cm and a light intensity of 67 mW.cm⁻² (±5%) between 230 and 400 nm (33 mWcm⁻² between 320 and 400 nm). During the irradiation, the Pyrex tube was rotated through one quarter of a turn each 5 minutes to allow a uniform bromination of the substrate. A flow of compressed air was used to keep the reaction vessel at room temperature. After an irradiation time of 20 min, the light source was switched off and the tube purged with nitrogen for 60 min. After that, the samples were removed from the tube and kept under vacuum for 24 h at room temperature to remove residual bromine.

Step 2: Grafting of Poly(2-hvdrmethyl Methacrylate) Brushes (PHEMA) from the Surface of the Brominated Hollow Fiber Membrane.

Surface-initiated atom transfer radical polymerization of HEMA was carried from the initiator coated PP hollow fiber following the method described in Example 1 (step 2) to form the PHEMA polymer brush coated membranes.

Step 3 (Protocol 1): Functionalization of the PHEMA Brushes with PBA Moieties

In a first strategy (protocol 1) the introduction of PBA groups into the PHEMA brush coating was carried following the method described in Example 3 (step 3 and 4).

Step 3 (Protocol 2): Functionalization of the PHEMA Brushes with PBA Moieties

In a second strategy (protocol 2), the introduction of PBA groups into the PHEMA brush coating was carried following the method described in Example 1 (step 2).

As detailed above, the process that was used for the modification of the PP hollow fiber substrate with PHEMA brushes started with the photobromination of the PP substrate, followed by SI-ATRP of HEMA using a CuCl/CuCl₂/bipy catalytic system. The bromination of the PP substrate was monitored with XPS (carried out as in Example 1). The XPS survey spectrum of the brominated PP hollow fiber surface (not shown) reveals the presence of the Br_{3d} (bromine), Br_{3p3/2} (bromine), Br_{3p1/2} (bromine) and Brₐₛ (bromine) signals along with a C₁ₛ (carbon) peak. The PHEMA modified PP substrates were characterized by XPS and ATR-FTIR spectroscopy (carried out as in example 1 and 3).

FIG. 16 shows the XPS survey spectra (left) and XPS C1s (carbon) core-level spectra (right) of: (A) unmodified PP hollow fiber; (B) PHEMA coated PP hollow fiber (outer part of the fiber); (C) PHEMA coated PP hollow fiber functionalized with carboxylic acid moieties; (D) PHEMA coated PP hollow fiber functionalized with phenylboronic acid moieties.

It is seen from FIG. 16 that the C₁ₛ (carbon) core-level spectrum recorded after a polymerization time of 15 min can be curve-fitted with five peak components at 285.0 eV, 285.8, 286.5, 287.3 and 289.3 eV respectively, which can be attributed to the aliphatic backbone C—C and C—H, terminal alcohol C—OH, ethylene glycol units (C—O) and ester groups (C═O—O) of PHEMA (FIG. 16B). The grafting of HEMA from the brominated PP substrates can also be conveniently monitored using ATR-FTIR spectroscopy (FIGS. 17B, 18B). The ATR-FTIR spectra of the PHEMA brushes show bands at 1726, 1270, 1251 and 1170 cm⁻¹, which can be attributed to C═O, C—O (ester), C—O (alcohol) and C—O (ether) stretching vibrations, respectively.

Two strategies were used to functionalize PHEMA brushes with PBA moieties (protocol 1 and 2). FIG. 17 shows ATR-FTIR spectra of the PP hollow fiber substrate as functionalized with PBA moieties according to protocol 1: (A) unmodified PP hollow fiber; (B) PHEMA coated PP hollow fiber; (C) PHEMA coated PP hollow fiber functionalized with carboxylic acid moieties; (D) PHEMA coated PP hollow fiber functionalized with phenylboronic acid moieties; (E) unmodified PP hollow fiber coated with 3-aminophenylboronic acid; (F) unmodified PP hollow fiber coated with (3-(dimethylamino)-1-propylamine. FIG. 18 shows ATR-FTIR spectra of the PP hollow fiber substrate as functionalized with PBA moieties according to protocol 2: (A) unmodified PP hollow fiber; (B) PHEMA coated PP hollow fiber; (C) PHEMA coated PP hollow fiber functionalized with NPC moieties; (D) PHEMA coated PP hollow fiber functionalized with phenylboronic acid moieties; (E) PHEMA coated PP hollow fiber functionalized with phenylboronic acid moieties and quenched with (3-(dimethylamino)-1-propylamine.

The process shown in FIG. 10 (protocol 1) is a two step strategy that involves the introduction of carbonyl groups and subsequent reaction with 3-aminophenyl boronic acid. The introduction of the carboxyl groups and attachment of the PBA moieties were confirmed by ATR-FTIR and XPS experiments. The XPS C1s (carbon) core level spectrum of the PHEMA brushes with carboxyl terminated side chains is shown in FIG. 16C. Two new peaks appear at 289.5 and 285.7 eV, which can be attributed to the aliphatic backbone (C—H) and ester groups (C═O—O) of the oxobutanoic acid moieties. The intensity of the carbonyl band at 1728 cm⁻¹ dramatically increases after the introduction of the oxobutanoic acid moieties (FIG. 17C). After conjugation with PBA, the XPS spectrum shows the appearance of a new carbonyl signal at 288.1 eV attributed to the presence of amide bond (FIG. 24D) and the XPS survey spectrum shows the presence of N1s (nitrogen) and B1s (boron) signals (FIG. 16D). The FTIR-ATR spectrum of the PBA functionalized PHEMA brushes shows the appearance of a new band at 1650 cm⁻¹ which confirms the presence of the amide bond (FIG. 17D).

The process shown in FIG. 4 (protocol 2) is a two step strategy that involves activation of the brush hydroxyl groups with p-nitrophenyl chloroformiate (NPC) and subsequent reaction with PBA. The attachment of the PBA group was confirmed by ATR-FTIR spectroscopy. As illustrated in FIG. 18, upon reaction of the NPC activated brush with PBA, the carbonyl band at 1770 cm⁻¹, which is due to the carbonate groups of the NPC activated brush, is replaced by two new bands at 1646 and 1532 cm⁻¹, which can be attributed to the amide vibrations. Glucose sensitivity of the PBA functionalized PHEMA polymer brush coating, has been demonstrated above (Example 2, FIGS. 14, 15).

1-19. (canceled)

20. A glucose responsive membrane for use in a medical device for the monitoring or regulation of glucose levels comprising:
a nanoporous support substrate, and
a glucose responsive hydrogel coating on a surface of the nanoporous substrate, said glucose responsive hydrogel coating comprising a polymeric matrix containing phenyl boronic acid functional groups, whereby the glucose responsive hydrogel is attached through covalent bonds or electrostatic interactions to the surface of the nanoporous substrate.

21. The membrane according to claim 20, wherein the hydrogel coating is attached to at least a part of the surface of the internal walls of pores of the nanoporous substrate.

22. The membrane according to claim 20, wherein the hydrogel coating has a thickness of no more than 300 nm.

23. The membrane according to claim 22, wherein the hydrogel coating has a thickness of from about 1 nm to about 200 nm.

24. The membrane according to claim 20, wherein the hydrogel coating comprises a plurality of polymer chains, at least a portion of which are functionalized with phenyl boronic acid functional groups, whereby each polymer chain is attached via one chain end thereof to a surface of the nanoporous substrate.

25. The membrane according to claim 20, wherein the hydrogel is formed by a controlled surface-initiated polymerisation process from the nanoporous substrate.

26. The membrane according to claim 20, wherein the glucose responsive hydrogel comprises 2-acrylamido-phenylboronic acid or 3-acrylamido-phenylboronic acid.

27. The membrane according to claim 20, wherein the glucose responsive hydrogel further comprises a tertiary or quaternary amino group.

28. The membrane according to claim 20, wherein the hydrogel comprises polymerised methacrylate, acrylate, methacrylamide, acrylamide or vinylic monomers.

29. The membrane according to claim 20, wherein the support substrate is a nanoporous polypropylene, nanoporous polyethylene or nanoporous alumina substrate.

30. The membrane according to claim 20, wherein the membrane comprises a biocompatible polymer, comprising anti-fouling functional groups, said biocompatible polymer being attached through covalent bonds to the glucose responsive hydrogel coating.

31. A method for the preparation of a glucose responsive membrane comprising:
covalently binding an initiator group to a surface of a nanoporous support substrate; and
subsequently forming a coating of a glucose responsive hydrogel comprising phenylboronic acid functional groups on the surface of the nanoporous substrate, via a controlled surface initiated polymerisation process from the initiator group.

32. The method according to claim 31 comprising a step of attaching a biocompatible polymer to the glucose responsive hydrogel via a controlled surface initiated polymerisation process of monomer groups funetionalised with anti-fouling moieties.

33. The method according to claim 31 for the preparation of a glucose responsive membrane comprising:
covalently binding an initiator group to a surface of a nanoporous support substrate; and
subsequently carrying out a polymerisation by a controlled surface initiated polymerisation process, from the initiator group, of monomers selected from methacrylate, acrylate, methacrylamide, acrylamide or vinylic monomers functionalised with phenylboronic acid functional groups; methacrylate, acrylate, methacrylamide, acrylamide or vinylic monomers functionalised with tertiary or quaternary amino groups; methacrylate, acrylate, methacrylamide, acrylamide or vinylic monomers functionalised with active ester groups; cross-linker groups;
and, optionally, monomers selected from acrylic or methacrylic monomers functionalised with 2-hydroethyl or polyethylene glycol moieties.

34. The method according to claim 31, wherein phenylboronic acid functional groups are introduced in the glucose responsive hydrogel coating by direct co-polymerisation of monomers comprising phenylboronic acid functional groups by a controlled surface initiated polymerisation process, from the initiator group.

35. The method according to claim 31, wherein phenylboronic acid functional groups are introduced in the glucose responsive hydrogel coating in a subsequent step by substitution of phenylboronic acid functional group containing moieties at activated sites in the formed hydrogel.

36. A medical device for the monitoring or regulation of glucose levels in a patient comprising:
an implantable member comprising a glucose responsive membrane, which reversibly changes its hydraulic permeability subject to changes in glucose concentration occurring in a medium surrounding the implantable member;
said membrane comprising a nanoporous support substrate; and
a biointerface configured to contact the medium surrounding the implantable member use, said biointerface comprising a glucose responsive hydrogel coating on the nanoporous substrate, said glucose responsive hydrogel coating comprising a polymeric matrix functionalised with phenyl boronic acid functional groups, whereby the glucose responsive hydrogel coating is attached through covalent bonds or electrostatic interactions to the surface of the nanoporous substrate.

37. The medical device according to claim 36 comprising a pressure generating means configured to deliver a liquid to the glucose responsive membrane; and a sensor adapted to measure a flow resistance of said liquid through the glucose responsive membrane; whereby glucose concentration is determined based on the flow resistance of the liquid through the glucose responsive membrane.

38. A method of monitoring or regulating blood glucose levels in a patient comprising contacting body fluid from a patient with a medical device comprising a glucose responsive membrane according to claim 20 and measuring blood glucose levels in said body fluid.
